(19) United States
(12) Patent Application Publication
(10) Pub. No.: US 2020/0085700 A1
(43) Pub. Date: Mar. 19, 2020

(54) SOLVENT MODIFICATIONS TO PREMOISTENED SUBSTRATES

(71) Applicant: GPCP IP Holdings LLC, Atlanta, GA (US)

(72) Inventors: Brian Patrick Argo, Greenville, WI (US); Abdus Salam, Harrisburg, NC (US); Jennifer L. Faucett, Green Bay, WI (US); Stephanie Petrina Boyce, Green Bay, WI (US); Kevin Matthew Holtman, Appleton, WI (US); Daniel Peter Gardner, Columbus, OH (US); Arinne Nicole Lyman, Greenville, WI (US)

(21) Appl. No.: 16/572,359

(22) Filed: Sep. 16, 2019

Related U.S. Application Data
(60) Provisional application No. 62/731,559, filed on Sep. 14, 2018.

(51) Int. Cl. A61K 8/02; A61K 8/20; A61K 8/34; A61K 8/92

(52) U.S. Cl. CPC A61K 8/0208; A61K 8/922; A61K 8/345; A61K 8/20

(57) ABSTRACT

Formulations suitable for use with fibrous substrates are provided. The formulations provide increased wet strength, high dispersibility, and shape retention of premoistened fibrous substrates. Such formulations comprise at least one dielectric-adjusting solvent, and optionally water, coupling agents, salts, personal care components, hard surface cleaning components, or combinations thereof.

### CROSS-REFERENCE TO RELATED APPLICATIONS

This application claims the benefit of priority to U.S. Provisional Application Ser. No. 62/731,559 filed on Sep. 14, 2018, the contents of which are hereby incorporated by reference herein in its entirety.

### 1. FIELD

The presently disclosed subject matter relates to formulations suitable for use with fibrous substrates and fibrous substrates including the same. Specifically, the formulations of the present disclosure provide for increased wet strength, high dispersibility, and shape retention of premoistened fibrous substrates. Such formulations comprise at least one dielectric-adjusting solvent, and optionally water, coupling agents, salts, personal care components, or hard surface cleaning components.

### 2. BACKGROUND

Formulations including solvents are used in a variety of applications, including personal care, institutional care, and cleaning products. In particular, such formulations can be used alone or in combination with other components to maintain dispersibility, strength, shape, and embossed patterns of fibrous substrates. For example, such formulations can be suitable for use with premoistened personal care wipes, such as baby wipes, adult perineal wipes, or facial wipes. These formulations can also be suitable for use with hard surface cleaning wipes.

Existing premoistened personal care wipes have areas of concern for consumers. For example, various wipes can have strength issues and tear apart in use due to not having proper wet strength when interacting with water. In addition, disposing of wipes in a sanitary manner can be challenging. Certain consumers may dispose of wipes in toilets, incorrectly assuming the dispersibility of the material. Other personal care wipes include amines and water-soluble binders which provide for sticky and unpleasant smelling wipes that are unacceptable for consumer use. Some personal care wipes are held together only by hydrogen bonding that degrades in water-based formulations resulting in wipes too weak for consumer use. Other personal care wipes include polymers that are not dispersible. The low dispersibility of current wipes causes hospitals and convalescence homes to dispose of perineal wipes with great expense as hazardous biowaste. Therefore, a strong, highly dispersible wipe is highly desired.

Current formulations include water-based or oil-based formulations. In water-based formulations, the polarity of water destroys hydrogen bonds of fibrous substrates and solubilizes water soluble binders providing a wipe that has little strength when used by a consumer. Thus, a water-based formulation provides wipes having acceptable cleaning properties with poor dispersibility or wet strength. In oil-based formulations, oil is non-polar and does not degrade fibrous substrates, however, oil is undesirable for cleansing applications because it smears rather than removes soils like fecal matter. Because oil does not readily disperse in water, an oil-coated substrate will not readily disperse in water. Thus, an oil-based formulation provides wipes having acceptable strength with poor dispersibility and poor cleaning properties. Accordingly, such formulations cannot provide an effective cleaning fibrous substrate having both increased wet strength and high dispersibility.

It is desirable, therefore, for formulations to provide premoistened wipes having increased wet strength, high dispersibility, shape retention, and effective cleaning properties suitable for consumer use. Thus, there remains a need in the art for formulations that are effective in providing increased wet strength, high dispersibility, and compatibility with raw materials beneficial to premoistened cleaning fibrous substrates. The presently disclosed subject matter addresses these and other needs.

### 3. SUMMARY OF THE INVENTION

The presently disclosed subject matter provides for formulations suitable for use with premoistened fibrous substrates and premoistened fibrous substrates including the same. It was surprisingly and advantageously found that the formulations disclosed herein provide for premoistened fibrous substrates having increased wet strength, high dispersibility, and shape retention in addition to compatibility with a wide variety of cleaning solutions. Such formulations comprise at least one dielectric constant-adjusting solvent, and optionally water, coupling agents, salts, personal care components, or hard surface cleaning components as discussed in further detail below. The dielectric constant (DEC) and decreased solubility of binders, such as carboxymethyl cellulose (CMC), of the formulations of the present disclosure surprisingly and advantageously provided for fibrous materials having both increased sheet strength and high dispersibility.

The presently disclosed subject matter provides for a dispersible wipe comprising a fibrous material and a lotion formulation. The lotion formulation has a dielectric constant of less than about 80. In certain embodiments, the lotion formulation can have a dielectric constant of between about 10 and about 60 or between about 30 and about 80.

In certain embodiments, the lotion formulation can include at least one solvent present in an amount of at least about 10 wt.-% and at least one salt present in an amount of at least about 0.5 wt.-%, based on the total weight of the lotion formulation. In certain embodiments, the at least one solvent can include butylene glycol, hexylene glycol, or combinations thereof and the at least one salt can include calcium chloride. In certain embodiments, the at least one solvent can be present in an amount of at least about 15 wt.-% with at least about 5 wt.-% hexylene glycol, based on the total weight of the lotion formulation. In certain embodiments, the lotion formulation can include butylene glycol present in an amount of at least about 30 wt.-% and the at least one salt can include calcium chloride.

In certain embodiments, the lotion formulation can comprise at least one solvent miscible with water having a dielectric constant or a Hansen Solubility Parameter with a lower polarity than water. In certain embodiments, the at least one solvent can comprise mineral oil, shea butter, cocoa butter, paraffin, beeswax, squalene, coconut oil, olive oil, cetyl alcohol, isopropyl myristate, triethylhexanoin, waxes, synthetic oils, plant oils, or combinations thereof.

In certain embodiments, the lotion formulation can comprise a solvent blend comprising at least one solvent miscible with water and at least one solvent immiscible with water. The solvent blend can be miscible with water.

In certain embodiments, the lotion formulation can comprise a solvent blend comprising water and at least one solvent miscible with water.

In certain embodiments, the lotion formulation can comprise at least one solvent comprising phenols, monohydric alcohols, diols, polyhydric alcohols, unsaturated aliphatic alcohols, alicyclic alcohols, glycols, glycol ethers, glycerin, glycol ethers, 3 propanediol, acetone, acetonitrile, or combinations thereof.

In certain embodiments, the lotion formulation can comprise at least one solvent present in an amount of from about 5 wt. % to about 80 wt. %, based on the total weight of the lotion formulation.

In certain embodiments, the lotion formulation can comprise at least one coupling agent. The at least one coupling agent can be a nonionic surfactant comprising polysorbate, alkylpolyglucosides, ethoxylated surfactants, sorbitan derivatives, betaines, amine oxides, or combinations thereof. The at least one coupling agent can be present in an amount of from about 0.1 wt. % to about 10 wt. %, based on the total weight of the lotion formulation.

In certain embodiments, the lotion formulation can comprise at least one fragrance. The at least one fragrance can be present in an amount of from about 0 wt. % to about 0.5 wt. %, based on the total weight of the lotion formulation.

In certain embodiments, the lotion formulation can comprise at least one preservative. The at least one preservative can be present in an amount of from about 0 wt. % to about 1.0 wt. %, based on the total weight of the lotion formulation.

In certain embodiments, the lotion formulation can comprise at least one skin protectant. The at least one skin protectant can be present in an amount of from about 0 wt. % to about 10 wt. %, based on the total weight of the lotion formulation.

In certain embodiments, the lotion formulation comprises at least one cationic disinfectant. The at least one cationic disinfectant can be present in an amount of from about 0 wt. % to about 1.0 wt. %, based on the total weight of the lotion formulation. In certain embodiments, the at least one cationic disinfectant can comprise alcohols, glycols, or a combination thereof present in an amount greater than about 50% by volume.

In certain embodiments, the lotion formulation comprises at least one detergent. The at least one detergent can be present in an amount of from about 0 wt. % to about 20 wt. %, based on the total weight of the lotion formulation.

The presently disclosed subject matter also provides for a dispersible wipe comprising a fibrous material, a water-soluble binder, and a lotion formulation. The lotion formulation has a dielectric constant of less than about 80. In certain embodiments, the lotion formulation can have a dielectric constant of between about 10 and about 60 or between about 30 and about 80.

In certain embodiments, the lotion formulation can include at least one solvent present in an amount of at least about 15 wt.-% and at least one salt present in an amount of at least about 0.5 wt.-%, based on the total weight of the lotion formulation. In certain embodiments, the at least one solvent can include butylene glycol and the at least one salt can include calcium chloride. In certain embodiments, the at least one solvent can be present in an amount of at least about 20 wt.-%, based on the total weight of the lotion formulation. In certain embodiments, the lotion formulation can include butylene glycol present in an amount of at least about 20 wt.-% and the at least one salt can include calcium chloride.

In certain embodiments, the lotion formulation can comprise at least one solvent miscible with water having a dielectric constant or a Hansen Solubility Parameter with a lower polarity than water. In certain embodiments, the at least one solvent can comprise mineral oil, shea butter, cocoa butter, paraffin, beeswax, squalene, coconut oil, olive oil, cetyl alcohol, isopropyl myristate, triethylhexanoin, waxes, synthetic oils, plant oils, or combinations thereof.

In certain embodiments, the lotion formulation can comprise a solvent blend comprising at least one solvent miscible with water and at least one solvent immiscible with water. The solvent blend can be miscible with water.

In certain embodiments, the lotion formulation can comprise a solvent blend comprising water and at least one solvent miscible with water.

In certain embodiments, the lotion formulation can comprise at least one solvent comprising phenols, monohydric alcohols, diols, polyhydric alcohols, unsaturated aliphatic alcohols, alicyclic alcohols, glycols, glycol ethers, glycerin, glycol ethers, 3 propanediol, acetone, acetonitrile, or combinations thereof.

In certain embodiments, the lotion formulation can comprise at least one solvent present in an amount of from about 5 wt. % to about 80 wt. %, based on the total weight of the lotion formulation.

In certain embodiments, the lotion formulation can comprise at least one coupling agent. The at least one coupling agent can be present in an amount of from about 0.1 wt. % to about 5 wt. %, based on the total weight of the lotion formulation.

In certain embodiments, the lotion formulation can comprise at least one fragrance. The at least one fragrance can be present in an amount of from about 0 wt. % to about 0.5 wt. %, based on the total weight of the lotion formulation.

In certain embodiments, the lotion formulation can comprise at least one disinfectant. The at least one disinfectant can be present in an amount of from about 0 wt. % to about 1.0 wt. %, based on the total weight of the lotion formulation. The at least one disinfectant can comprise alcohols, glycols, or a combination thereof present in an amount greater than about 50% by volume.

In certain embodiments, the lotion formulation can comprise at least one detergent. The at least one detergent can be present in an amount of about 0 wt. % to about 20 wt. %, based on the total weight of the lotion formulation.

In certain embodiments, the lotion formulation comprises at least one salt. The at least one salt can comprise a cation comprising sodium, potassium, calcium, magnesium, iron, aluminum, potassium, silver, tin, zinc, ammonium, or combinations thereof and anions selected from the group consisting of chloride, phosphate, sulfate, nitrite, or nitrate. In certain embodiments, the at least one salt is calcium chloride.

The presently disclosed subject matter also provides for a lotion formulation suitable for use with fibrous substrates. The lotion formulation comprises at least one solvent and has a dielectric constant of less than about 80.

The presently disclosed subject matter also provides for a kit, comprising a nonwoven substrate and the lotion formulation provided herein. The lotion formulation has a dielectric constant of less than about 80.

The foregoing has outlined broadly the features and technical advantages of the present application in order that the detailed description that follows may be better understood. Additional features and advantages of the application will be described hereinafter which form the subject of the claims of the application. It should be appreciated by those skilled in the art that the conception and specific embodiment disclosed may be readily utilized as a basis for modifying or designing other structures for carrying out the same purposes of the present application. It should also be realized by those skilled in the art that such equivalent constructions do not depart from the spirit and scope of the application as set forth in the appended claims. The novel features which are believed to be characteristic of the application, both as to its organization and method of operation, together with further objects and advantages will be better understood from the following description.

### 4. BRIEF DESCRIPTION OF THE DRAWINGS

FIG. 1 depicts a graph showing the CDW and MDW tensile strength of samples including various solvents in accordance with Example 1 to test solvent effects on hydrogen bonding. The graph shows the CDW and MDW tensile strength (y-axis) versus the solvent included in the sample (x-axis). For each sample, the left column represents the CDW tensile strength (% of dry) and the right column represents the MDW tensile strength (% of dry).

FIG. 2 depicts a graph showing the CDW and MDW tensile strength increase over water of samples including various solvents blended with a tissue base sheet in accordance with Example 1 to test solvent effects on hydrogen bonding. The graph shows the CDW and MDW tensile strength increase over water (y-axis) versus the solvent included in the sample (x-axis). For each sample, the left column represents the CDW tensile strength increase over water (%) and the right column represents the MDW tensile strength increase over water (%).

FIG. 3 depicts a graph showing the CDW and MDW tensile strength and dielectric constant of samples in accordance with Example 1 to test solvent effects on hydrogen bonding. The graph shows the CDW and MDW tensile strength (y-axis) versus the dielectric constant (x-axis) and illustrates the dielectric constant effect on tensile retention.

FIG. 4 depicts a graph showing the ball burst force of samples including various solvents in accordance with Example 1 to test solvent effects on binder dissolution. The graph shows the ball burst force (y-axis) versus the solvent concentration (x-axis) and illustrates the effects of solvents mixed with water on ball burst force. Line (A) represents the ball burst force (lb.) results of the glycerin-based sample. Line (B) represents the ball burst force (lb.) results of the butylene glycol-based sample. Line (C) represents the ball burst force (lb.) results of the dipropylene glycol monomethyl ether-based sample.

FIGS. 5A-5C depict contour plots showing the ball burst force of samples including various lotions as provided in Example 7. FIG. 5A provides the calcium chloride and butylene glycol concentration versus ball burst force at 2.00% carboxymethyl cellulose (CMC). FIG. 5B provides the calcium chloride and butylene glycol concentration versus ball burst force at 2.45% carboxymethyl cellulose (CMC). FIG. 5C provides the calcium chloride and butylene glycol concentration versus ball burst force at 2.90% carboxymethyl cellulose (CMC).

FIGS. 6A-6C depict contour plots showing ball burst force—dry retention (% dry) of samples including various lotions as provided in Example 7. FIG. 6A provides the calcium chloride and butylene glycol concentration versus ball burst force—dry retention (% dry) at 2.00% carboxymethyl cellulose (CMC). FIG. 6B provides the calcium chloride and butylene glycol concentration versus ball burst force—dry retention (% dry) at 2.45% carboxymethyl cellulose (CMC). FIG. 6C provides the calcium chloride and butylene glycol concentration versus ball burst force—dry retention (% dry) at 2.90% carboxymethyl cellulose (CMC).

FIGS. 7A-7C depict contour plots showing CDW tensile strength of samples including various lotions as provided in Example 8. FIG. 7A provides the calcium chloride and butylene glycol concentration versus CDW tensile strength at 2.00% carboxymethyl cellulose (CMC). FIG. 7B provides the calcium chloride and butylene glycol concentration versus CDW tensile strength at 2.45% carboxymethyl cellulose (CMC). FIG. 7C provides the calcium chloride and butylene glycol concentration versus CDW tensile strength at 2.90% carboxymethyl cellulose (CMC).

FIGS. 8A-8C depict contour plots showing MDW tensile strength of samples including various lotions as provided in Example 8. FIG. 8A provides the calcium chloride and butylene glycol concentration versus MDW tensile strength at 2.00% carboxymethyl cellulose (CMC). FIG. 7B provides the calcium chloride and butylene glycol concentration versus MDW tensile strength at 2.45% carboxymethyl cellulose (CMC). FIG. 7C provides the calcium chloride and butylene glycol concentration versus MDW tensile strength at 2.90% carboxymethyl cellulose (CMC).

FIG. 9 depicts a graph showing the average percent solids after filtering relative to carboxymethyl cellulose (CMC) added in order of increasing calcium chloride concentration for various lotions in accordance with Example 11.

FIG. 10 depicts a graph showing the average percent solids after filtering relative to carboxymethyl cellulose (CMC) added in order of increasing butylene glycol concentration for various lotions in accordance with Example 11.

FIGS. 11A-11C depict graphs showing the carboxymethyl cellulose (CMC) filtrate (% of original CMC) for 0.5% calcium chloride and 15% butylene glycol; 30% butylene glycol; and 45% butylene glycol, respectively, for various lotions in accordance with Example 11. FIG. 11A provides the carboxymethyl cellulose (CMC) filtrate (% of original CMC) for 0.5% calcium chloride and 15% butylene glycol. FIG. 11B provides the carboxymethyl cellulose (CMC) filtrate (% of original CMC) for 0.5% calcium chloride and 30% butylene glycol. FIG. 11C provides the carboxymethyl cellulose (CMC) filtrate (% of original CMC) for 0.5% calcium chloride and 45% butylene glycol.

FIGS. 12A-12C depict graphs showing the carboxymethyl cellulose (CMC) filtrate (% of original CMC) for 2.5% calcium chloride and 15% butylene glycol; 30% butylene glycol; and 45% butylene glycol, respectively, for various lotions in accordance with Example 11. FIG. 12A provides the carboxymethyl cellulose (CMC) filtrate (% of original CMC) for 2.5% calcium chloride and 15% butylene glycol. FIG. 12B provides the carboxymethyl cellulose (CMC) filtrate (% of original CMC) for 2.5% calcium chloride and 30% butylene glycol. FIG. 12C provides the carboxymethyl cellulose (CMC) filtrate (% of original CMC) for 2.5% calcium chloride and 45% butylene glycol.

### 5. DETAILED DESCRIPTION

The presently disclosed subject matter relates to formulations suitable for use with fibrous substrates and fibrous substrates including the same. Specifically, the formulations of the present disclosure uniquely provide for increased wet strength, high dispersibility, and shape retention of premoistened fibrous substrates. The dielectric constant (DEC) and binder solubility of such formulations surprisingly and advantageously provided for fibrous substrates having both high wet strength and high dispersibility.

The presently disclosed subject matter provides formulations suitable for use with fibrous substrates, such as personal care wipes, that provide for fibrous substrates that disperse rapidly into individual fibers. Due to having a lower dielectric constant than water, solvents included in formulations of the present disclosure can preserve hydrogen bonds and therefore maintain the embossed patterns and shape of fibrous substrates providing for wipes having improved cleaning and brand recognition. Formulations of the presently disclosed subject matter also allow for use of a wide variety of base sheets with little or no binder, which significantly reduces base sheet costs. Overall product costs are also easily minimized by balancing the polarity of such formulations against the strength of the individual base sheet.

Formulations of the presently disclosed subject matter can affect dispersible fibrous substrates in several ways. If dispersible fibrous substrates have a high dry strength primarily due to hydrogen bonds, the majority of the dry strength can be preserved in a wipe that disperses instantaneously when exposed to water. Formulations of the present disclosure can also reduce the solubility of water-soluble binders. As the polarity of the formulation decreases, the solubility of the binder decreases, and the base sheet strengthens. In addition, in reducing the solubility of binders, such formulations of the present disclosure can magnify the effects of salts so that low levels of salt are used to reduce solubility of binders further in a very economical, skin-friendly manner.

These and other aspects of the disclosed subject matter are discussed more in detail in this description and below in the Examples.

### A. Definitions

The terms used in this specification generally have their ordinary meanings in the art, within the context of this subject matter and in the specific context where each term is used. Certain terms are defined below to provide additional guidance in describing the compositions and methods of the disclosed subject matter and how to make and use them.

As used in the specification and the appended claims, the singular forms “a,” “an,” and “the” include plural referents unless the context clearly dictates otherwise. Thus, for example, reference to “a compound” includes mixtures of compounds.

The term “about” or “approximately” means within an acceptable error range for the particular value as determined by one of ordinary skill in the art, which will depend in part on how the value is measured or determined, i.e., the limitations of the measurement system. For example, “about” can mean within three or more than three standard deviations, per the practice in the art. Alternatively, “about” can mean a range of up to 20%, preferably up to 10%, more preferably up to 5%, and more preferably still up to 1% of a given value. Also, particularly with respect to systems or processes, the term can mean within an order of magnitude, preferably within five-fold, and more preferably within two-fold, of a value.

As used herein, the term “dielectric constant” refers to the ratio of the permittivity of a substance to the permittivity of free space, a relative measure of chemical polarity. Dielectric constants for pure substances are easily measured, however, modelled dielectric constants are difficult to estimate as provided in Jouyban, et al., International Journal of Pharmaceutics, Vol. 269 (2) (2004) 353-360, which is incorporated herein by reference in its entirety.

As used herein, the term “weight percent” is meant to refer to the quantity by weight of a constituent or component in the formulation as a percentage of the overall weight of the formulation. The terms “weight percent,” “w %,” “wt. %”, and “wt %” are used interchangeably.

### B. Formulation Components

The presently disclosed subject matter relates to formulations suitable for use with fibrous substrates. Specifically, the formulations comprise at least one dielectric-adjusting solvent, and optionally water, coupling agents, salts, personal care components, or hard surface cleaning components.

### i. Solvents

Formulations of the present disclosure comprise at least one dielectric-adjusting solvent. In certain embodiments, the solvent can have a lower dielectric constant than water. In certain embodiments, the solvent can be miscible with water. In certain embodiments, the formulation can include a solvent that has a lower dielectric constant than water and is miscible with water. In an alternative embodiment, the solvent can be immiscible with water.

The presently disclosed formulations can include one or more solvent. As used herein, a “solvent blend” also refers to a blend including one or more solvents. In certain embodiments, the formulation can include a solvent blend including at least one solvent miscible with water and at least one solvent immiscible with water. The solvent immiscible with water can be present in relatively low amounts so that the solvent blend remains miscible with water. The solvent immiscible with water can significantly reduce the dielectric constant of the solvent blend.

Solvents suitable for use in the formulations of the present disclosure are provided in Table 1.

TABLE 1

| | | Dielectric | Solubility in | | |
|---|---|---|---|---|---|
| | | Constant | Water per 100 g | Acceptable in | Flash Point |
| Solvent | Tradename | (DEC) | at RT | Cosmetics | (° C.) |
| Water | — | 80.1 | Miscible | Yes | 75 |
| Propylene carbonate* | Jeffsol ® | 64.0 | 24 | Yes | 121 |
| | Propylene | | | | |
| | carbonate | | | | |
| Glycerin | — | 46.5 | Miscible | Yes | 160 |
| Xylitol | OriStar XLT | 40.0 | Miscible | Yes | — |
| Ethylene Glycol phenyl | Dowanol ™ | 37.7 | 2.5 | Yes | 119 |
| ether | EPh | | | | |
| Isopentyldiol | Isoprene Glycol | 37.7 | Miscible | Perfume | 63 |
| Sorbitol | — | 35.5 | — | — | — |
| 1,3 propane diol | Zemea ® | 35.0 | Miscible | Yes | 92 |
| Propylene Glycol | — | 32.0 | Miscible | Yes | 104 |
| Dipropylene Glycol | — | — | Miscible | Yes | 99 |
| Butylene Glycol | — | 28.8 | Miscible | Yes | 116 |
| D-Mannitol | — | 24.6 | — | — | — |
| Ethanol* | — | 25.3 | Miscible | Yes | 17 |
| Lactic Acid | — | 22.0 | — | — | — |
| Acetone* | — | 21.0 | — | — | — |
| Isopropyl Alcohol* | — | 18.5 | Miscible | Perfume | 81 |
| Pentanol | — | 15.1 | Miscible | — | — |
| Hexanol | — | 13.0 | 0.59 | Yes | 235 |
| Dipropylene Glycol | Dowanol ™ | 10.5 | Miscible | Yes | 135 |
| Monomethyl Ether* | DPM | | | | |
| 2-Butoxyethanol* | ButylCellosolve | 9.4 | Miscible | — | — |
| Hexylene Glycol* | — | 7.7 | Miscible | Yes | 93 |
| Acetic Acid* | — | 6.2 | — | — | — |
| Ethyl Acrylate | — | 6.1 | — | — | — |
| Polyvinyl alcohol in | — | ~2.5 | — | — | — |
| Water | | | | | |
| Mineral Oil | — | 2.1 | Immiscible | Yes | 160 |
| Vegetable Oil (e.g., | — | 2.95 | Immiscible | Yes | 160 |
| corn oil; cottonseed oil) | | | | | |
| Silicone Oil | — | 2.75 | Immiscible | Yes | 160 |

*Suitable for hard surface cleaners

In certain embodiments, the solvent or solvent blend can be undiluted with water. The dielectric constant of the solvent or solvent blend can be lower than water, which has a dielectric constant of about 80.1. The solvent or solvent blend can have a total dielectric constant between about 1 and about 80, between about 5 and about 60, between about 10 and about 60, between about 30 and about 80, between about 1 and about 75, between about 2 and about 70, or between about 3 and about 65. In certain embodiments, the dielectric constant of the solvent or solvent blend can be less than about 80, less than about 75, or less than about 60. In particular embodiments, the dielectric constant of the solvent or solvent blend can be about 5, about 10, about 15, about 25, about 30, about 45, about 50, about 55, about 60, about 65, about 70, about 75, or about 80.

In certain embodiments, the solvent or solvent blend can be diluted with water to form a solvent-water blend. In such solvent-water blends, the dielectric constant of the solvent or solvent blend can have a dielectric constant of between about 10 and about 75, about 30 and about 70, or about 40 and about 65.

In certain embodiments, the solvent or solvent blend can be diluted with water and at least one salt to form a solvent, water and salt blend. While the solvent can reduce the dielectric constant, the addition of salt increases the dielectric constant of the formulation. In certain embodiments, the blend can have a dielectric constant of between about 10 and about 75, between about 30 and about 70, or between about 40 and about 65. In certain embodiments, the solvent or solvent blend can be diluted with water prior to the addition of one or more salts. In certain solvent-water blends, the total blend can have a dielectric constant of between about 10 and about 75, between about 30 and about 70, or between about 40 and about 65, prior to the addition of one or more salts.

In certain embodiments, the solvent or solvent blend can be diluted with personal care components such as pH modifiers, humectants, emollients, skin protectants, polymers, coupling agents, mild cleaners, or combinations thereof. The overall dielectric constant of these formulations can be between about 10 and about 95, between about 30 and about 70, or between about 40 and about 70.

In certain embodiments, the solvent or solvent blend can be diluted with hard surface cleaning components such as builders, salts, surfactants, or combinations thereof. While the at least one solvent can reduce the dielectric constant, the addition of builders and surfactants can increase the dielectric constant of the formulation. In certain embodiments, the solvent or solvent blend can be diluted with water prior to the addition of builders, salts, surfactants, or combinations thereof. In solvent-water blends, the total mixture can have a dielectric constant of between about 10 and about 75, about 30 and about 70, or about 40 and about 65 prior to the addition of builders, salts, surfactants, or combinations thereof.

In certain embodiments, Hansen Solubility Parameters can be used to describe low polarity, miscible solvents. Hansen Solubility Parameters are listed in Hansen, C., Hansen Solubility Parameters, A User's Handbook, 2nd Edition, 2007, which is incorporated herein by reference in its entirety. Hansen Solubility Parameters use three parameters: dispersion (δ_{D}), polarity (δ_{P}), and hydrogen-bonding (δ_{H}). Each parameter is equally viable to modify a formulation. Parameters can be modified individually or jointly so long as the formulation is miscible with water, preserves hydrogen bonding, and reduces binder solubility. Every solvent does not need to be miscible with water, so long as the blend is readily miscible with water. Immiscible solvents can reduce the Hansen Solubility Parameters and when combined with a sufficient quantity of miscible solvents are acceptable for use with dispersible premoistened wipes.

In certain embodiments, the solvent or solvent blend can be combined with a substrate in the absence of water. In certain embodiments, the solvent or solvent blends can be readily miscible with water, without the presence of water. Some of the solvents in the blend can be immiscible solvents and can therefore shift the dielectric constant disproportionately. Thus, immiscible solvents can be mixed with a miscible solvent at a sufficiently low concentration so that the solvent blend is miscible with water. If wipe coatings are not sufficiently miscible with water, the dispersibility of the wipe slows accordingly.

In certain embodiments, the solvent or solvent blend is mixed with water prior to application to a fibrous substrate. The solvent or solvent blend can be miscible with water. Solvent blends can contain high levels of water as long as the dielectric constant of the final solution is sufficient to maintain the hydrogen bonding of the fibers and/or reduce the solubility of the binder. Some solvents, like isopropyl alcohol can be used at about 100% concentration and blended with water with fibrous substrates strengthened by hydrogen bonding. However, water-solvent blends including solvents such as isopropyl alcohol are not appropriate for use with water-soluble binders as isopropyl alcohol will rapidly separate when used on substrate that contains small amounts of salt or solubilized binder.

In certain embodiments, at least one solvent or solvent blend that is salt stable and miscible can be mixed with water and salt and added to a fibrous substrate with a water-soluble binder to reduce the water solubility of the binder and strengthen the base sheet.

In certain embodiments, a solvent or solvent blend can be mixed with water and personal care components such as pH adjusters, preservatives, builders, botanical agents, colorants, fragrances, surfactants, emollients, skin protectants and a dispersible fibrous substrate.

In certain embodiments, a solvent or solvent blend can be mixed with water and hard surface cleaning components such as pH adjusters, preservatives, builders, surface protectants, colorants, fragrances, surfactants, disinfectants, and a dispersible fibrous substrate.

In certain embodiments, a solvent can have a flash point in a range of from about 10° C. to about 200° C. In certain embodiments, at least one solvent can have a low flash point. For example, in certain embodiments, a solvent can have a flash point in a range of from about 10° C. to about 37° C. In alternative embodiments, a solvent can have a high flash point in the range of from about 40° C. to about 200° C.

All solvents suitable for use in the present disclosure reduce the solubility of binders and protect the hydrogen bonding of fibers by reducing the dielectric constant or modifying the Hansen Solubility Parameters of formulations of the present disclosure. In some embodiments, a solvent or solvent blend can be present in an amount of from about 1 wt. % to about 100 wt. %, based on the overall weight of the formulation. In some embodiments, the solvent or solvent blend can be present in an amount of from about 5 wt. % to about 50 wt. %, from about 5 wt. % to about 35 wt. %, or from about 5 wt. % to about 10 wt. %, based on the overall weight of the formulation. In particular embodiments, the solvent or solvent blend can be present in an amount of about 5 wt. %, about 10 wt.-%, about 25 wt. %, about 50 wt. %, about 75 wt. %, about 90 wt. %, or about 99 wt. %, based on the overall weight of the formulation. In certain embodiments, the solvent or solvent blend can be present in an amount of at least about 10 wt.-%, at least about 15 wt.-%, at least about 20 wt.-%, or at least about 30 wt.-%, based on the overall weight of the formulation.

In certain embodiments, the solvent or solvent blend can include butylene glycol, hexylene glycol, or combinations thereof. In particular embodiments, the solvent or solvent blend can include hexylene glycol present in an amount of at least about 10 wt.-%, based on the overall weight of the formulation. In particular embodiments, the solvent or solvent blend can include butylene glycol present in an amount of at least about 15 wt.-% and hexylene glycol present in an amount of at least about 5 wt.-%, based on the overall weight of the formulation. In particular embodiments, the solvent or solvent blend can include butylene glycol present in an amount of at least about 10 wt.-%, at least about 15 wt.-%, at least about 20 wt.-%, or at least about 30 wt.-%, based on the overall weight of the formulation.

In certain embodiments, the solvent or solvent blend can include any miscible solvent with a lower dielectric constant than water. Suitable solvents include, but are not limited to, phenols, monohydric alcohols, diols, polyhydric alcohols, unsaturated aliphatic alcohols, alicyclic alcohols, glycols, glycol ethers, glycerin, glycol ethers, 3 propanediol, acetone, acetonitrile, and combinations thereof. Glycol ethers include Butyl Carbitol, Dowanol™ DPM, or other Dowanol™ solvents (Dow Chemical, Midland, Mich., USA), 1 Alcohols include methanol, ethanol, isopropyl alcohol, n-butyl alcohol, t-butyl alcohol, or n-hexanol. Diols and polyols include propylene glycol, butylene glycol, hexylene glycol, glycerin, and 1,3 propane diol. A person of skill in the art will appreciate a wide variety of solvent dielectric constant adjusters are suitable for use in the formulations presently disclosed.

Formulations of the present disclosure can also include immiscible solvents, so long as such solvents are present in an amount that is sufficiently low that the formulation applied to the fibrous substrate remains miscible with water. Immiscible solvents can have lower dielectric constants than miscible solvents. Thus, a solvent blend including miscible solvents and immiscible solvents can reduce the total dielectric constant below a formulation including miscible solvents. Suitable immiscible solvents include, but are not limited to, mineral oil, shea butter cocoa butter, paraffin, beeswax, squalene, coconut oil, olive oil, cetyl alcohol, isopropyl myristate, triethylhexanoin, waxes, synthetic oils, and other plant oils. Immiscible solvents or miscible solvents with lower dielectric constants reduce the amount of total solvent needed to drive the total system dielectric constant to a minimum level.

### ii. Coupling Agents

The presently disclosed formulations can further comprise at least one coupling agent. In certain embodiments, the at least one coupling agent can include polyoxylene sorbitan fatty acid derivatives (BASF, Florham Park, N.J.), such as Polysorbate 20, Polysorbate 40, Polysorbate 60, and Polysorbate 80, Triton® BG-10, Triton® CG-50, Triton® CG-600, Triton® CG-650, Triton® CG-11-alkyl poly glucosides (Dow Chemical, Midland, Mich.), Plantacare® 1200 UP, Plantacare® 2000 UP, Plantacare® 820 UP, Plantacare® 818 UP, Plantapon® LGC Sorb and Texapon® EASY (BASF, Florham Park, N.J.), Tergitol™ 15-S-7, Tergitol™ 15-S-9, Tergitol™ 15-S-15, Tergitol™ 15-S-20, Tergitol™ 15-S-30, Tergitol™ TMN 6, Tergitol™ TMN 10 (Dow Chemical, Midland, Mich.), Tomamine Amphoteric L (Evonik, Allentown, Pa.), PEG cetyl/oleyl ethers such as Teric™ 17A3, Teric™ 17A8 (Huntsman Performance Products, The Woodlands, Tex.), or combinations thereof. A person of skill in the art will appreciate a wide variety of coupling agents are suitable for use in the formulations of the present disclosure.

The coupling agent can be present in an amount of from about 0 wt. % to about 20 wt. %, about 0 wt. % to about 10 wt. % or about 0.1 wt. % to about 10 wt. %, based on the overall weight of the formulation. In some embodiments, the coupling agent can be present in an amount of from about 0 wt. % to about 5 wt. % or about 0.1 wt. % to about 5 wt. %, based on the overall weight of the formulation. In some embodiments, the coupling agent can be present in an amount from about 0 wt. % to about 2 wt. %, based on the overall weight of the formulation. In particular embodiments, the coupling agent can be present in an amount of about 0.1 wt.-%, about 0.5 wt. %, about 1 wt. %, about 5 wt. %, about 8 wt. %, about 10 wt. %, about 15 wt. %, or about 20 wt. %, based on the overall weight of the formulation.

### iii. Salts

The presently disclosed formulations can further comprise one or more salts. In certain embodiments, the one or more salts can include cations such as sodium, potassium, calcium, magnesium, zinc, copper (I) or copper (II), tin (II) or tin (IV), ammonium, aluminum, iron (II) or iron (III), and anions such as hydroxide, chloride, fluoride, iodide, bromide sulfate, sulfite, phosphate, carbonate, citrate, nitrate, acetate or any salt deemed safe and effective for the application. For example, and not by limitation, the one or more salts can include sodium chloride (NaCl), calcium chloride (CaCl₂)), and combinations thereof, although a person of skilled in the art will appreciate that a wide variety of salts are suitable for use in the formulations of the present disclosure.

In certain embodiments, the one or more salts can be present in the amount of from about 0 wt. % to about 10 wt. %, based on the overall weight of the formulation. In certain embodiments, the one or more salts can be present in an amount of from about 0 wt. % to about 4 wt. % or from about 0 wt. % to about 2 wt. %, based on the overall weight of the formulation. In particular embodiments, the one or more salts can be present in an amount of about 0.5 wt. %, about 1 wt. %, about 1.5 wt. %, about 2 wt.-%, about 2.5 wt. %, about 3 wt. %, or about 4 wt. %, based on the overall weight of the formulation. In certain embodiments, the one or more salts can be present in an amount of at least about 0.5 wt.-%, at least about 1 wt.-%, or at least about 1.5 wt.-%, based on the overall weight of the formulation.

Although salts are polar substances, salts can be used in formulations of the present disclosure having reduced dielectric constants or Hansen Solubility Parameters. Without being limited by a particular theory, in solvent-water blends and fibrous substrates that include dispersible binders, salts can synergistically work with the solvent-water blend to increase wet strength while maintaining dispersibility. Salts can reduce the solubility of the binder and the solvent-water blend also reduces the solubility of the binder. Because salt reduces free water activity, less water is available to dissolve the binder. Additionally, multivalent ions can replace sodium ions in binders like sodium carboxymethyl cellulose (CMC), further reducing binder solubility. Reducing the polarity of the formulation can increase the effect of salt in reducing binder solubility.

### iv. Personal Care Components

The presently disclosed formulations can further include one or more components for personal care applications. In certain embodiments, the one or more personal care components can include preservatives, fragrances, skin protectants such as humectants and/or emollients, thickeners, sequestering agents, pH adjusters, and combinations thereof. However, a person of skill in the art will appreciate that various personal care components, e.g., those commonly used in the art in formulations suitable for use with fibrous substrates, can also be present.

### Preservatives

In certain embodiments, the formulation can include one or more preservatives. Preservatives typically reduce the dielectric constant of the formulation. In some embodiments, glycol water formulations can be self-preserving. In other embodiments, the formulations can require preservatives. Any suitable preservative can be incorporated into the formulations of the present disclosure, including combinations or blends thereof. Preservatives can include a mixture of organic acids and alcohols, which further increases the decrease of the dielectric constant of the presently disclosed formulations.

In certain embodiments, the one or more preservatives can include phenoxy ethanol (Optiphen PO, Ashland, Bridgewater, N.J.), benzyl alcohol/Dehydroacetic acid/benzoic acid (Microcare® BPD, Thor Personal Care, Compiègne, France), benzyl alcohol Dehydroacetic acid/benzoic acid (Microcare® BDB, Thor Personal Care, Compiègne, France), dehydroacetic acid (Geogard 111, Lonza, Basel, Switzerland), phenoxy ethanol/ethylhexylglycerin (Euxyl® PE 9010, Shülke, Norderstadt, Germany), phenoxy ethanol/caprylyl glycol (Optiphen 200, Ashland, Bridgewater, N.J.), phenoxy ethanol/caprylyl glycol/Decylene glycol (Microcare® PDHG2, Thor Personal Care, Compiègne, France), 1,2 Hexanediol/phenyl propanol (Microcare® APHX, Thor Personal Care, Compiègne, France), Xylitol esters/caprylyl glycol (Hebeatol CG, Chemyunion, Sao Paulo, Brazil), butylene glycol/benzyl alcohol/sorbic acid/caprylic triglyceride/capric triglyceride/lauryl alcohol/Myristyl alcohol (Geogard LSA, Lonza, Basel, Switzerland), Kathon CG (Dow Chemical, Midland, Mich.), or combinations thereof. A person of skill in the art will appreciate a wide variety of preservatives are suitable for use in the formulations of the present disclosure.

In certain embodiments, the one or more preservatives can be present in an amount of from about 0 wt. % to about 2.0 wt. %, based on the overall weight of the formulation, depending on the preservative used. In some embodiments, the one or more preservatives can be present in an amount of from about 0.2 wt. % to about 1.6 wt. %, about 0.5 wt. % to about 1.5 wt. %, or about 0.5 wt. % to about 1.0 wt. %, based on the overall weight of the formulation. In particular embodiments, the one or more preservatives can be present in an amount of about 0.2 wt. %, about 0.5 wt. %, about 0.8 wt. %, about 1 wt. %, about 1.5 wt. %, about 1.6 wt. %, about 1.8 wt. %, or about 2 wt. %, based on the overall weight of the formulation.

### Fragrances

In certain embodiments, the formulation can include one or more fragrances. The use of a fragrance can enhance consumer experience by providing a premoistened fibrous substrate with a pleasant smell. In certain embodiments, the one or more fragrances can include a cosmetic grade fragrance. A person of skill in the art will appreciate that a wide variety of fragrances are suitable for use in the formulations of the present disclosure.

The one or more fragrances can be present in an amount of from about 0 wt. % to about 0.5 wt. %, based on the overall weight of the formulation. In some embodiments, the one or more fragrances can be present in an amount of from about 0 wt. % to about 0.2 wt. % or about 0 wt. % to about 0.1 wt. %, based on the overall weight of the formulation. In certain embodiments, the one or more fragrances can be present in an amount of about 0 wt. % to about 0.05 wt. %, based on the total weight of the formulation. In particular embodiments, the one or more fragrances can be present in an amount of 0.05 wt. %, about 0.08 wt. %, about 0.1 wt. %, about 0.2 wt. %, about 0.3 wt. %, about 0.4 wt. %, or about 0.5 wt. %, based on the overall weight of the formulation.

### Skin Protectants

In certain embodiments, the formulation can include one or more skin protectants. The one or more skin protectants can include humectants to preserve moisture on the skin. Such humectants include water-soluble sugars. Water-soluble sugars can further reduce the dielectric constant of the formulation. Suitable water-soluble sugars include, for example, glucose, galactose, fructose, mannose, sucrose, or combinations thereof. In some embodiments, humectants can be present in an amount of from about 0 wt. % to about 10 wt. %, about 0 wt. % to about 2 wt. %, or about 0 wt. % to about 0.5 wt. %, based on the overall weight of the formulation. In particular embodiments, humectants can be present in an amount of 0.5 wt. %, about 1 wt. %, about 1.5 wt. %, about 2 wt. %, or about 5 wt. %, based on the overall weight of the formulation. In some embodiments, the one or more skin protectants can include emollients which soften skin in creating an occlusive protective barrier. Suitable emollients include, for example, mineral oil, shea butter, cocoa butter, mineral oil, paraffin, beeswax, squalene, coconut oil, olive oil, cetyl alcohol, isopropyl myristate, triethylhexanoin, plant oils, or combinations thereof. Since these emollients have dielectric constants about 3, the overall dielectric constant of the formulation can be reduced. In certain embodiments, emollients can be added in small amounts to maintain miscibility of the formulation. In some embodiments, emollients can be present in an amount of from about 0 wt. % to about 5 wt. %, about 0 wt. % to about 1 wt. %, or about 0 wt. % to about 0.5 wt. %, based on the overall weight of the formulation. In particular embodiments, emollients can be present in an amount of about 0.05 wt. %, about 0.1 wt. %, about 0.2 wt. %, about 0.5 wt. %, about 1 wt. %, about 1.5 wt. %, about 2 wt. %, or about 5 wt. %, based on the overall weight of the formulation.

In some embodiments, the one or more skin protectants can include cocamidopropyl PG-dimonium chloride phosphate (Cola® Lipid C) (Colonial Chemical, South Pittsburgh, Tenn.), silicone, Bis-PEG-18 methyl ether dimethyl silane (Gransil VX-406), film forming surfactants, or combinations thereof. In certain embodiments, the one or more skin protectants can include, for example, lauroyl lysine, ethylene glycol disterate (EGDS), polydimethylsiloxane (PDMS, silicone fluid, various viscosities), vegetable oils (e.g., coconut oil, avocado oil, or olive oil), Dowsil EP 9801 hydro cosmetic powder (Dimethicone/vinyl dimethicone crosspolymer and silica and butylene glycol), fatty esters and blends, esterquats (e.g., Rewoquat WE 45), or combinations thereof. In certain embodiments, one or more skin protectants can be present in an amount of from about 0 wt. % to about 15 wt. %, about 0 wt. % to about 5 wt. %, or about 0 wt. % to about 2 wt. %, based on the overall weight of the formulation. In some embodiments, one or more skin protectants including silicone can be present in an amount of from about 0 wt. % to about 10 wt. %, about 0 wt. % to about 5 wt. %, or about 0 wt. % to about 2 wt. %, based on the overall weight of the formulation. In some embodiments, one or more skin protectants including cocamidopropyl PG-dimonium chloride phosphate can be present in an amount of from about 0 wt. % to about 10 wt. %, about 0 wt. % to about 5 wt. %, or about 0 wt. % to about 2 wt. %, based on the overall weight of the formulation. In particular embodiments, the one or more skin protectants can be present in an amount of about 0.5 wt. %, about 1 wt.-%, about 2 wt. %, about 5 wt. %, about 8 wt. %, or about 10 wt. %, based on the overall weight of the formulation. A person of skill in the art will appreciate a wide variety of skin protectants are suitable for use in formulations of the present disclosure.

### Thickeners

In certain embodiments, the formulation can include one or more thickeners. Thickeners can be used to reduce fluid migration in a fibrous substrate and reduce the dielectric constant of the formulation. Miscible dielectric constant adjusters can be polymers such as polyvinyl alcohol, polyacrylamide, polyethylene glycol, polyvinyl pyrrolidone, polyurethane, or polyvinyl methyl ether/maleic anhydride. Polyvinyl alcohol has a dielectric constant of about 2.5, and polyethylene glycol has a dielectric constant of about 10 (Carbowax 1000 EE, Dow Chemical, Midland, Mich.). In some embodiments, one or more thickeners can be present in an amount of from about 0 wt. % to about 2 wt. %, about 0 wt. % to about 0.5 wt. %, or about 0 wt. % to about 0.1 wt. %, based on the overall weight of the formulation. In particular embodiments, the one or more thickeners can be present in an amount of 0.5 wt. %, about 0.1 wt. %, about 0.2 wt. %, about 0.5 wt. %, about 1 wt. %, about 1.5 wt. %, or about 2 wt. %, based on the overall weight of the formulation. A person of skill in the art will appreciate a wide variety of thickeners are suitable for use in formulations of the present disclosure.

### Sequestering Agents

In certain embodiments, the formulation can include one or more sequestering agents. In certain embodiments, the formulation can include one or more metal sequestering agents. Sequestering agents can minimize the impact of hard water during commercial blending operations. Sequestering agents can be used in absence of multivalent ions and can aid in surfactant performance and preservation. The one or more sequestering agents can include, but are not limited to, salts of ethylenediaminetetraacetic acid (EDTA), sodium phytate/phytic acid, sodium citrate/citric acid, sodium gluconate, nitrilotriacetic acid, trisodium ethylenediamine dissucinate, and combinations thereof. In some embodiments, one or more sequestering agents can be present in an amount of from about 0 wt. % to about 0.5 wt. %, about 0 wt. % to about 0.3 wt. %, or about 0 wt. % to about 0.1 wt. %, based on the overall weight of the formulation. In certain embodiments, one or more sequestering agents including ethylenediaminetetraacetic acid (EDTA) can be present in an amount of from about 0 wt. % to about 0.5 wt. %, about 0 wt. % to about 0.3 wt. %, or about 0 wt. % to about 0.1 wt. %, based on the overall weight of the formulation. In particular embodiments, the one or more sequestering agents can be present in an amount of 0.1 wt. %, about 0.2 wt. %, about 0.3 wt. %, about 0.4 wt. %, or about 0.5 wt. %, based on the overall weight of the formulation. A person of skill in the art will appreciate a wide variety of sequestering agents are suitable for use in the formulations of the present disclosure.

### pH Adjusters

In certain embodiments, the formulation can include one or more pH adjusters. The one or more pH adjusters can include a base such as sodium or potassium hydroxide. The one or more pH adjusters can also include an organic acid. Organic acids can reduce the dielectric constant of the formulation. Organic acids suitable for use in the presently disclosed formulations include acetic acid, ascorbic acid, lactic acid, malic acid, maleic acid, succinic acid, tartaric acid, glycolic acid, citric acid, and combinations thereof. In some embodiments, the one or more pH adjusters can include lactic acid. The one or more pH adjusters can be added in a quantity sufficient to adjust the formulation to a pH of from about 3 to about 10, about 3.5 to about 7, or about 4 to about 6. A person of skill in the art will appreciate a wide variety of pH adjusters are suitable for use in formulations of the present disclosure.

### v. Hard Surface Cleaning Components

The presently disclosed formulations can further include one or more components for hard surface cleaning applications. In certain embodiments, the one or more hard surface cleaning components can include preservatives, fragrances, detergents, builders such as sequestering agents, pH adjusters, disinfectants, and combinations thereof. However, a person of skill in the art will appreciate that various hard surface cleaning components, e.g., those commonly used in the art in formulations suitable for use with fibrous substrates, can also be present.

### Preservatives

In certain embodiments, the formulation can include one or more preservatives. Preservatives typically reduce the dielectric constant of the formulation. In some embodiments, glycol water formulations can be self-preserving. Other formulations can require preservatives. Any suitable preservative can be incorporated into the formulations of the present disclosure, including combinations or blends thereof. Preservatives can include a mixture of organic acids and alcohols, which further increases the decrease of the dielectric constant of the presently disclosed formulations.

In certain embodiments, the one or more preservatives can include phenoxy ethanol (Optiphen PO, Ashland, Bridgewater, N.J.), benzyl alcohol/Dehydroacetic acid/benzoic acid (Microcare® BPD, Thor Personal Care, Compiègne, France), benzyl alcohol Dehydroacetic acid/benzoic acid (Microcare® BDB, Thor Personal Care, Compiègne, France), dehydroacetic acid (Geogard 111, Lonza, Basel, Switzerland), phenoxy ethanol/ethylhexylglycerin (Euxyl® PE 9010, Shülke, Norderstadt, Germany), phenoxy ethanol/caprylyl glycol (Optiphen 200, Ashland, Bridgewater, N.J.), phenoxy ethanol/caprylyl glycol/Decylene glycol (Microcare® PDHG2, Thor Personal Care, Compiègne, France), 1,2 Hexanediol/phenyl propanol (Microcare® APHX, Thor Personal Care, Compiègne, France), Xylitol esters/caprylyl glycol (Hebeatol CG, Chemyunion, Sao Paulo, Brazil), butylene glycol/benzyl alcohol/sorbic acid/caprylic triglyceride/capric triglyceride/lauryl alcohol/Myristyl alcohol (Geogard LSA, Lonza, Basel, Switzerland), 2-methyl-4isothiazolin-3-one/2-n-octyl-4-isothiazolin-3-one/glycol esters (Bioban 425, Dow Chemical, Midland, Mich.), or combinations thereof. A person of skill in the art will appreciate a wide variety of preservatives are suitable for use in the formulations of the present disclosure.

In certain embodiments, the one or more preservatives can be present in the amount of from about 0 wt. % to about 2.0 wt. %, based on the overall weight of the formulation, depending on the preservative used. In some embodiments, the one or more preservatives can be present in an amount of from about 0.2 wt. % to about 1.6 wt. %, about 0.5 wt. % to about 1.5 wt. %, about 0.5 wt. % to about 1.0 wt. %, or about 0.01 wt. % to about 0.075 wt. %, based on the overall weight of the formulation. In particular embodiments, the one or more preservatives can be present in an amount of about 0.01 wt. %, about 0.1 wt. %, about 0.2 wt. %, about 0.5 wt. %, about 0.8 wt. %, about 1 wt. %, about 1.5 wt. %, about 1.6 wt. %, about 1.8 wt. %, or about 2 wt. %, based on the overall weight of the formulation.

### Fragrances

In certain embodiments, the formulation can include one or more fragrances. The use of a fragrance can enhance consumer experience by providing a premoistened fibrous substrate with a pleasant smell. In certain embodiments, the one or more fragrances can include Aloe Green Floral M1 RTB-00434. A person of skill in the art will appreciate that a wide variety of fragrances are suitable for use in the formulations of the present disclosure.

The one or more fragrances can be present in an amount of from about 0 wt. % to about 0.5 wt. %, based on the overall weight of the formulation. In some embodiments, the one or more fragrances can be present in an amount of from about 0 wt. % to about 0.05 wt. %, about 0 wt. % to about 0.02 wt. %, or about 0 wt. % to about 0.05 wt. %, based on the overall weight of the formulation. In particular embodiments, the one or more fragrances can be present in an amount of 0.05 wt. %, about 0.08 wt. %, about 0.1 wt. %, about 0.2 wt. %, about 0.3 wt. %, about 0.4 wt. %, or about 0.5 wt. %, based on the overall weight of the formulation.

### Detergents

In certain embodiments, the formulation can include one or more detergents. Detergents can provide increased cleaning performance of premoistened fibrous substrates. The one or more detergents can include zwitterionic detergents such a lauramine oxide (Ammonyx LO, Stepan, Northfield, Ill.), anionic surfactants such as sodium laureth sulfate (Steol CS-330, Stepan, Northfield, Ill.) or sodium lauryl sulfate (Stepanol WA-loo NF, Stepan, Northfield, Ill.), nonionic surfactants such as alkyl polyglucosides (Triton® BG-10, Dow Chemical, Midland, Mich. or Triton® CG-50, Dow Chemical, Midland, Mich.), or combinations thereof. The one or more detergents can be present in an amount of from about 0 wt. % to about 20 wt. %, based on the overall weight of the formulation. In some embodiments, the one or more detergents can be present in an amount of from about 0 wt. % to about 0.5 wt. % or about 0 wt. % to about 0.2 wt. %, based on the overall weight of the formulation. In particular embodiments, the one or more detergents can be present in an amount of about 0.01 wt. %, about 0.05 wt. %, about 0.1 wt. %, about 1.5 wt. %, or about 0.2 wt. %, based on the overall weight of the formulation. A person of skill in the art will appreciate that a wide variety of detergents are suitable for use in the formulations of the present disclosure.

### pH Adjusters

In certain embodiments, the formulation can include one or more pH adjusters. The one or more pH adjusters can include a base such as sodium or potassium hydroxide, an organic acid, or an inorganic acid. Organic acids can reduce the dielectric constant of the formulation. Organic acids suitable for use in the presently disclosed formulations include acetic acid, ascorbic acid, lactic acid, malic acid, maleic acid, succinic acid, tartaric acid, glycolic acid, citric acid, and combinations thereof. Inorganic acids suitable for use in the presently disclosed formulations include hydrochloric acid, sulfuric acid, nitric acid, phosphoric acid, and combinations thereof pH limits can be determined by the cleaning application and stability of the substrate. In certain embodiments, the one or more pH adjusters can be added in a quantity sufficient to adjust the formulation to a pH of from about 3 to about 12, about 4 to about 11, or about 4.5 to about 10.5. A person of skill in the art will appreciate that a wide variety of pH adjusters are suitable for use in the formulations of the present disclosure.

### Builders

In certain embodiments, the formulation can include one or more builders. The one or more builders can include one or more sequestering agents. In certain embodiments, the formulation can include one or more metal sequestering agents. Sequestering agents can minimize the impact of hard water during commercial blending operations. Sequestering agents can be used in absence of multivalent ions and can aid in surfactant performance and preservation. In certain embodiments, the one or more builders can include, but are not limited to, salts of ethylenediaminetetraacetic acid (EDTA), sodium phytate/phytic acid, sodium citrate/citric acid, sodium gluconate, nitrilotriacetic acid, trisodium ethylenediamine dissucinate, sodium carbonate, and combinations thereof. In some embodiments, one or more builders can be present in an amount of from about 0 wt. % to about 0.5 wt. %, about 0 wt. % to about 0.3 wt. %, or about 0 wt. % to about 0.1 wt. %, based on the overall weight of the formulation. In some embodiments, one or more builders including ethylenediaminetetraacetic acid (EDTA) can be present in an amount of from about 0 wt. % to about 0.5 wt. %, about 0 wt. % to about 0.3 wt. %, or about 0 wt. % to about 0.1 wt. %, based on the total weight of the formulation. In particular embodiments, the one or more builders can be present in an amount of about 0.1 wt. %, about 0.2 wt. %, about 0.3 wt. %, about 0.4 wt. %, or about 0.5 wt. %, based on the overall weight of the formulation. A person of skill in the art will appreciate a wide variety of builders are suitable for use in the formulations of the present disclosure.

### Disinfectants

In certain embodiments, the formulation can include one or more disinfectants. Presently disclosed formulations including alcohol can also serve as a disinfectant. Suitable alcohols include, for example, ethanol, isopropyl alcohol, biguanides, phenols, essential oils, or combinations thereof. Additionally, quaternary ammonium chloride compounds such as benzalkonium chloride (Bardac 205 M & Bardac 208M, Lonza, Basel, Switzerland), benzethonium chloride (Lonzagard, Lonza, Basel, Switzerland) can be added in some embodiments. Additionally, general disinfectants as provided in Block, Seymore (Ed.), Disinfection, Sterilization, and Preservation, 5th Edition, Lippincott Williams & Wilkins, 2001, which is incorporated by reference herein in its entirety, can be suitable for use in formulations of the present disclosure. The one or more disinfectants can be present in an amount of from about 0 wt. % to about 1.0 wt. %, about 0 wt. % to about 0.5 wt. %, or about 0 wt. % to about 0.3 wt. %, based on the overall weight of the formulation. In particular embodiments, the one or more disinfectants can be present in an amount of about 0.01 wt. %, about 0.05 wt. %, about 0.1 wt. %, about 0.2 wt. %, about 0.3 wt. %, about 0.5 wt. %, or about 1 wt. %, based on the overall weight of the formulation. Cationic disinfectants can be consumed by certain base sheets, and thus a quantity of cationic disinfectants to compensate for adsorption to the base sheet can be added. Solvents including alcohols, glycols, or combinations thereof at greater than about 50% by volume or greater than about 59% by volume can also be suitable disinfectants. The range of cationic surfactants refers to the level of surfactant measurable in solution extracted from a base sheet. A person of skill in the art will appreciate a wide variety of disinfectants are suitable for use in the formulations of the present disclosure.

### C. Formulations

The presently disclosed subject matter relates to formulations suitable for use with fibrous substrates. As used herein, the term “formulation” is used interchangeably with the term “lotion” or “lotion formulation”. Specifically, the formulations of the present disclosure provide increased wet strength, high dispersibility, and shape retention of premoistened fibrous substrates. Such formulations comprise at least one dielectric-adjusting solvent. In certain embodiments, the at least one dielectric-adjusting solvent can have a dielectric constant lower than water and can be miscible with water. The synergistic effect of the dielectric constant and solubility of binders of such formulations, surprisingly and advantageously provided fibrous substrates with both high wet strength and high dispersibility.

Formulations of the present disclosure can optionally include one or more additional components, including, but not limited to, immiscible solvents, water, coupling agents, salts, personal care components, or hard surface cleaning components. Personal care components can include preservatives, fragrances, skin protectants such as humectants and/or emollients, thickeners, sequestering agents, pH adjusters, and combinations thereof. Hard surface cleaning components can include preservatives, fragrances, detergents, builders such as sequestering agents, pH adjusters, disinfectants, and combinations thereof. The formulation can be suitable for use with fibrous substrates, such as premoistened personal care wipes or hard surface cleaning wipes.

The formulation can be an aqueous or non-aqueous solution. In certain embodiments, the formulation is an aqueous solution. In certain embodiments, the formulation is a non-aqueous solution. Aqueous formulations can have any suitable pH range. For example, and not by limitation, the pH of the formulation can range from about 3.0 to about 10.0, from about 4.0 to about 8.0, or from about 4.0 to about 6.0 for personal care products. In certain embodiments, for personal care products, the formulation can have a pH of about 4, about 4.5, about 5, about 5.2, about 6, about 7, or about 8. For example, and not by limitation, the pH of the formulation can range from about 2.0 to about 12.0, from about 3.0 to about 11.0, or from about 4.0 to about 11.0 for hard surface cleaning products. In certain embodiments, for hard surface cleaning products, the formulation can have a pH of about 2, about 3, about 4, about 5, about 6, about 7, about 8, about 9, about 10, about 11, or about 12. Since pH is a measure of conductivity in water, formulations including pure solvents of the present disclosure do not have a pH.

In certain embodiments, the formulation can include water, at least one dielectric-adjusting solvent, a coupling agent, a skin protectant, a chelating agent, a preservative, and a fragrance. In particular embodiments, the formulation can further include one or more salts. In certain embodiments, the formulation can include water present in an amount of quantum satis (Q.S.), at least one dielectric-adjusting solvent present in an amount of from about 1 wt.-% to about 100 wt.-%, a coupling agent present in an amount of from about 0 wt.-% to about 10 wt.-%, a skin protectant present in an amount of from about 0 wt.-% to about 10 wt.-%, a chelating agent present in an amount of from about 0 wt.-% to about 0.5 wt.-%, a preservative present in an amount of from about 0 wt.-% to about 2 wt.-%, and a fragrance present in an amount of about 0 wt.-% to about 0.5 wt.-%, based on the total weight of the formulation. The formulation can further include one or more salts present in an amount of from about 0 wt.-% to about 10 wt.-%, based on the total weight of the formulation.

In certain embodiments, the formulation can include water, at least one dielectric-adjusting solvent, a coupling agent, a skin protectant, a skin cleanser, a preservative, and a fragrance. In particular embodiments, the formulation can further include a sheet strengthener, one or more salts, or a combination thereof. In certain embodiments, the formulation can include water present in an amount of quantum satis (Q. S.), at least one dielectric-adjusting solvent present in an amount of from about 1 wt.-% to about 100 wt.-%, a coupling agent present in an amount of from about 0 wt.-% to about 10 wt.-%, a skin protectant present in an amount of from about 0 wt.-% to about 10 wt.-%, a chelating agent present in an amount of from about 0 wt.-% to about 0.5 wt.-%, a preservative present in an amount of from about 0 wt.-% to about 2 wt.-%, and a fragrance present in an amount of about 0 wt.-% to about 0.5 wt.-%, based on the total weight of the formulation. The formulation can further include one or more salts present in an amount of 0 wt.-% to about 10 wt.-%, a sheet strengthener present in an amount of from about 0 wt.-% to about 10 wt.-%, or combinations thereof, based on the total weight of the formulation.

In certain embodiments, the formulation can include water, at least one dielectric-adjusting solvent, a sheet strengthener, a coupling agent, a preservative, and a fragrance. In particular embodiments, the formulation can further include one more salts. In certain embodiments, the formulation can include water present in an amount of quantum satis (Q.S.), at least one dielectric-adjusting solvent present in an amount of from about 1 wt.-% to about 100 wt.-%, a sheet strengthener present in an amount of from about 0 wt.-% to about 10 wt.-%, a coupling agent present in an amount of from about 0 wt.-% to about 20 wt.-%, a preservative present in an amount of from about 0 wt.-% to about 2 wt.-%, and a fragrance present in an amount of about 0 wt.-% to about 0.5 wt.-%, based on the total weight of the formulation. The formulation can further include one or more salts present in an amount of from about 0 wt.-% to about 10 wt.-%, based on the total weight of the formulation.

In certain embodiments, the formulation can include water (Q. S.), a dielectric-adjusting solvent (approx. 50 wt.-%), a coupling agent (approx. 0.50 wt.-%), a skin protectant (approx. 1.50 wt.-%), a chelating agent (approx. 0.10 wt.-%), a preservative (approx. 0.50 wt.-%), and a fragrance (approx. 0.05 wt.-%), based on the total weight of the formulation. The water can be deionized water. The dielectric-adjusting solvent can include propylene glycol or butylene glycol. The coupling agent can include Polysorbate 20. The skin protectant can include Cola® Lipid C. The chelating agent can include ethylenediaminetraacetic acid (EDTA). The preservative can include Microcare® BDP. In certain embodiments, the formulation can further include one or more salts present in an amount of about 1 wt.-%, about 2 wt.-%, or about 4 wt.-%, based on the total weight of the formulation. The one or more salts can include sodium chloride.

In certain embodiments, the formulation can include water (Q.S.), a first dielectric-adjusting solvent (approx. 50 wt.-%), a second dielectric-adjusting solvent (approx. 10 wt.-%), a coupling agent (approx. 0.50 wt.-%), a skin protectant (approx. 1.50 wt.-%), a chelating agent (approx. 0.10 wt.-%), a preservative (approx. 0.50 wt.-%), and a fragrance (approx. 0.05 wt.-%), based on the total weight of the formulation. The water can be deionized water. The first dielectric-adjusting solvent can include propylene glycol or butylene glycol. The second dielectric-adjusting solvent can include glycerin. The coupling agent can include Polysorbate 20. The skin protectant can include Cola® Lipid C. The chelating agent can include ethylenediaminetraacetic acid (EDTA). The preservative can include Microcare® BDP.

In certain embodiments, the formulation can include water (Q.S.), a first dielectric-adjusting solvent (approx. 50 wt.-%), a second dielectric-adjusting solvent (approx. 20 wt.-%), a coupling agent (approx. 0.50 wt.-%), a skin protectant (approx. 1.50 wt.-%), a chelating agent (approx. 0.10 wt.-%), a preservative (approx. 0.50 wt.-%), and a fragrance (approx. 0.05 wt.-%), based on the total weight of the formulation. The water can be deionized water. The first dielectric-adjusting solvent can include propylene glycol or butylene glycol. The second dielectric-adjusting solvent can include glycerin. The coupling agent can include Polysorbate 20. The skin protectant can include Cola® Lipid C. The chelating agent can include ethylenediaminetraacetic acid (EDTA). The preservative can include Microcare® BDP.

In certain embodiments, the formulation can include water (Q.S.), a first dielectric-adjusting solvent (approx. 50 wt.-%), a second dielectric-adjusting solvent (approx. 30 wt.-%), a coupling agent (approx. 0.50 wt.-%), a skin protectant (approx. 1.50 wt.-%), a chelating agent (approx. 0.10 wt.-%), a preservative (approx. 0.50 wt.-%), and a fragrance (approx. 0.05 wt.-%), based on the total weight of the formulation. The water can be deionized water. The first dielectric-adjusting solvent can include propylene glycol or butylene glycol. The second dielectric-adjusting solvent can include glycerin. The coupling agent can include Polysorbate 20. The skin protectant can include Cola® Lipid C. The chelating agent can include ethylenediaminetraacetic acid (EDTA). The preservative can include Microcare® BDP.

In certain embodiments, the formulation can include water (Q.S.), a first dielectric-adjusting solvent (approx. 50 wt.-%), a second dielectric-adjusting solvent (approx. 10 wt.-%), a coupling agent (approx. 0.50 wt.-%), a skin protectant (approx. 1.50 wt.-%), a preservative (approx. 0.50 wt.-%), and a fragrance (approx. 0.05 wt.-%), based on the total weight of the formulation. The water can be deionized water. The first dielectric-adjusting solvent can include butylene glycol. The second dielectric-adjusting solvent can include glycerin. The coupling agent can include Polysorbate 20. The skin protectant can include Cola® Lipid C. The preservative can include Microcare® BDP. In certain embodiments, the formulation can further include one or more salts present in an amount of about 0.50 wt.-%, about 1 wt.-%, about 2 wt.-%, or about 4 wt.-%, based on the total weight of the formulation. The one or more salts can include calcium chloride.

In certain embodiments, the formulation can include water (Q. S.), a dielectric-adjusting solvent (approx. 50 wt.-%), a coupling agent (approx. 0.50 wt.-%), a skin protectant (approx. 1.50 wt.-%), a preservative (approx. 0.50 wt.-%), and a fragrance (approx. 0.05 wt.-%), based on the total weight of the formulation. The water can be deionized water. The dielectric-adjusting solvent can include butylene glycol. The coupling agent can include Polysorbate 20. The skin protectant can include Cola® Lipid C. The preservative can include Microcare® BDP. In certain embodiments, the formulation can further include a sheet-strengthening agent present in an amount of about 0.05 wt.-%, based on a total weight of the formulation. The sheet-strengthening agent can include calcium chloride dihydrate. In certain embodiments, the formulation can further additionally include one or more salts present in an amount of about 1.50 wt.-%, based on the total weight of the formulation. The one or more salts can include sodium chloride.

In certain embodiments, the formulation can include water (Q.S.), a first dielectric-adjusting solvent (approx. 50 wt.-%), a second dielectric-adjusting solvent (approx. 15 wt.-%), a coupling agent (approx. 0.50 wt.-%), a skin protectant (approx. 1.50 wt.-%), a preservative (approx. 0.50 wt.-%), and a fragrance (approx. 0.05 wt.-%), based on the total weight of the formulation. In certain embodiments, the formulation can include a fragrance present in an amount of about 0.5 wt.-%, based on the total weight of the formulation. The first dielectric-adjusting solvent can include butylene glycol. The second dielectric-adjusting solvent can include dipropylene glycol monomethyl ether (Dowanol™ DPM). The coupling agent can include Polysorbate 20. The skin protectant can include Cola® Lipid C. The preservative can include Microcare® BDP. In certain embodiments, the formulation can further include a sheet-strengthening agent present in an amount of about 0.05 wt.-%, based on the total weight of the formulation. The sheet-strengthening agent can include calcium chloride dihydrate. In certain embodiments, the formulation can further include one or more salts present in an amount of about 1.50 wt.-%, based on the total weight of the formulation. The one or more salts can include sodium chloride. In certain embodiments, the formulation can further include a sheet-strengthening agent (approx. 0.05 wt.-%) and one or more salts (approx. 1.50 wt.-%), based on the total weight of the formulation. The sheet-strengthening agent can include calcium chloride dihydrate. The one or more salts can include sodium chloride.

In certain embodiments, the formulation can include water (Q.S.), a first dielectric-adjusting solvent (approx. 50 wt.-%), a second dielectric-adjusting solvent (approx. 5 wt.-%), a coupling agent (approx. 0.50 wt.-%), a skin protectant (approx. 1.50 wt.-%), a preservative (approx. 0.50 wt.-%), and a fragrance (approx. 0.05 wt.-%), based on the total weight of the formulation. The water can be deionized water. The first dielectric-adjusting solvent can include butylene glycol. The second dielectric-adjusting solvent can include hexylene glycol. The coupling agent can include Polysorbate 20. The skin protectant can include Cola® Lipid C. The preservative can include Microcare® BDP. In certain embodiments, the formulation can further include one or more salts present in an amount of about 1.50 wt.-%, based on the total weight of the formulation. The one or more salts can include sodium chloride. Alternatively, in certain embodiments, the formulation can further include a sheet-strengthening agent present in an amount of about 0.50 wt.-%, based on the total weight of the formulation. The sheet-strengthening agent can include calcium chloride dihydrate. Alternatively, in certain embodiments, the formulation can further include one or more salts present in an amount of about 0.50 wt.-%, about 1 wt.-%, about 1.50 wt.-%, about 2 wt.-%, about 2.50 wt.-%, about 2.50 wt.-%, or about 4 wt.-%, based on the total weight of the formulation. The one or more salts can include calcium chloride.

In certain embodiments, the formulation can include water (Q.S.), a first dielectric-adjusting solvent (approx. 50 wt.-%), a second dielectric-adjusting solvent (approx. 10 wt.-%), a coupling agent (approx. 0.50 wt.-%), a skin protectant (approx. 1.50 wt.-%), a preservative (approx. 0.50 wt.-%), and a fragrance (approx. 0.05 wt.-%), based on the total weight of the formulation. The water can be deionized water. The first dielectric-adjusting solvent can include butylene glycol. The second dielectric-adjusting solvent can include hexylene glycol. The coupling agent can include Polysorbate 20. The skin protectant can include Cola® Lipid C. The preservative can include Microcare® BDP. In certain embodiments, the formulation can further include one or more salts present in an amount of about 1.50 wt.-%, based on the total weight of the formulation. The one or more salts can include sodium chloride. Alternatively, in certain embodiments, the formulation can further include a sheet-strengthening agent present in an amount of about 0.50 wt.-%, based on the total weight of the formulation. The sheet-strengthening agent can include calcium chloride dihydrate. Alternatively, in certain embodiments, the formulation can further include one or more salts present in an amount of about 0.50 wt.-%, about 1 wt.-%, or about 1.50 wt.-%, based on the total weight of the formulation. The one or more salts can include calcium chloride.

In certain embodiments, the formulation can include water (Q.S.), a first dielectric-adjusting solvent (Q.S.), a second dielectric-adjusting solvent (approx. 5 wt.-%), a sheet-strengthening agent (approx. 2.50 wt.-%), a coupling agent (approx. 0.50 wt.-%), a preservative (approx. 0.50 wt.-%), and a fragrance (approx. 0.05 wt.-%), based on the total weight of the formulation. The water can be deionized water. The first dielectric-adjusting solvent can include butylene glycol. The second dielectric-adjusting solvent can include hexylene glycol. The sheet-strengthening agent can include calcium chloride dihydrate. The coupling agent can include Polysorbate 20. The preservative can include Microcare® BDP. Alternatively, in certain embodiments, the first dielectric-adjusting solvent can be present in an amount of about 20 wt.-%, about 30 wt.-%, about 40 wt.-%, or about 50 wt.-%, based on the total weight of the formulation.

In certain embodiments, the formulation can include water (Q.S.), a first dielectric-adjusting solvent (approx. 30 wt.-%), a second dielectric-adjusting solvent (approx. 5 wt.-%), a sheet-strengthening agent (Q.S.), a coupling agent (approx. 0.50 wt.-%), a preservative (approx. 0.50 wt.-%), and a fragrance (approx. 0.05 wt.-%), based on the total weight of the formulation. In certain embodiments, the formulation can further include one or more salts present in an amount of about 0.37 wt.-%, about 0.90 wt.-%, about 1.70 wt.-%, or about 2.50 wt.-%, based on the total weight of the formulation. The one or more salts can include calcium chloride.

In certain embodiments, the formulation can include a solvent, a first dielectric-adjusting solvent, a second dielectric-adjusting solvent, a coupling agent, a preservative, and one or more salts. The formulation can further include a fragrance.

In certain embodiments, the formulation can include water (Q.S.). The formulation can further include a first dielectric-adjusting solvent present in an amount of about 15 wt.-%, about 30 wt.-%, or about 45 wt.-%, based on the total weight of the formulation. The formulation can further include a second dielectric-adjusting solvent (approx. 5 wt.-%), a coupling agent (approx. 0.50 wt.-%), and a preservative (approx. 0.80 wt.-%). The formulation can further include one or more salts present in an amount of about 0.5 wt.-%, 1.5 wt.-%, or about 2.5 wt.-%, based on the total weight of the formulation. The water can be deionized water. The first dielectric-adjusting solvent can include butylene glycol. The second dielectric-adjusting solvent can include hexylene glycol. The coupling agent can include Polysorbate 20. The preservative can include phenoxy ethanol. The one or more salts can include calcium chloride. In certain embodiments, the formulation can further include a fragrance. The fragrance can be present in an amount of about 0.05 wt.-%, based on the total weight of the formulation.

In certain embodiments, the formulation can include water (approx. 45.34 wt.-%). The formulation can include a first dielectric-adjusting solvent present in an amount of about 0 wt.-%, about 15 wt.-%, about 30 wt.-%, or about 45 wt.-%, based on the total weight of the formulation. The formulation can further include a second dielectric-adjusting solvent (approx. 5 wt.-%), a coupling agent (approx. 0.50 wt.-%), and a preservative (approx. 0.80 wt.-%). The formulation can further one or more salts present in an amount of about 0 wt.-%, about 0.5 wt.-%, about 1 wt.-%, or about 2.5 wt.-%, based on the total weight of the formulation. The water can be deionized water. The first dielectric-adjusting solvent can include butylene glycol. The second dielectric-adjusting solvent can include hexylene glycol. The coupling agent can include Polysorbate 20. The preservative can include phenoxy ethanol. The one or more salts can include calcium chloride. In certain embodiments, the formulation can further include a fragrance. The fragrance can be present in an amount of about 0.05 wt.-%, based on the total weight of the formulation. In certain embodiments, the formulation can further include sodium carboxymethyl cellulose (CMC).

### D. Fibrous Substrates

Formulations of the present disclosure are suitable for use with fibrous substrates. Fibrous substrates of the present disclosure can be used for any application as known in the art. Such fibrous substrates can be used alone or as a component in other consumer products. For example, fibrous substrates of the present disclosure can be used alone or as a component in a variety of articles including hard surface cleaning wipes, personal care wipes, paper towels, tissues, toilet paper, and the like. In certain embodiments, the fibrous substrates can include natural fibers, synthetic fibers, or combinations thereof. In certain embodiments, the fibrous substrates can include one or more layers. In certain embodiments, the fibrous substrates can be nonwoven materials. The fibrous substrates of the present subject matter can be dispersible.

### Cellulose Fibers

Any cellulose fibers known in the art, including cellulose fibers of any natural origin, such as those derived from wood pulp or regenerated cellulose, can be used in fibrous substrates of the present disclosure. In certain embodiments, cellulose fibers include, but are not limited to, digested fibers, such as kraft, prehydrolyzed kraft, soda, sulfite, chemi-thermal mechanical, and thermo-mechanical treated fibers, derived from softwood, hardwood or cotton linters. In other embodiments, cellulose fibers include, but are not limited to, kraft digested fibers, including prehydrolyzed kraft digested fibers.

Non-limiting examples of cellulose fibers suitable for use in this subject matter are the cellulose fibers derived from softwoods, such as pines, firs, and spruces. Other suitable cellulose fibers include, but are not limited to, those derived from Esparto grass, bagasse, kemp, flax, hemp, kenaf, and other lignaceous and cellulosic fiber sources. Suitable cellulose fibers include, but are not limited to, bleached Kraft southern pine fibers sold under the trademark FOLEY FLUFFS® (available from GP Cellulose).

The fibrous substrates of the disclosed subject matter can also include, but are not limited to, commercially available bright fluff pulp including, but not limited to, southern softwood kraft (such as Golden Isles® 4725 from GP Cellulose) or southern softwood fluff pulp (such as Treated FOLEY FLUFFS® or Golden Isles® 4723 from GP Cellulose), northern softwood sulfite pulp (such as T 730 from Weyerhaeuser), or hardwood pulp (such as eucalyptus). While certain pulps can be preferred based on a variety of factors, any cellulosic fluff pulp or mixtures thereof can be used. In certain embodiments, wood cellulose, cotton linter pulp, chemically modified cellulose such as cross-linked cellulose fibers and highly purified cellulose fibers can be used. Non-limiting examples of additional pulps are FOLEY FLUFFS® FFTAS (also known as FFTAS or GP Cellulose FFT-AS pulp), and Weyco CF401.

### Synthetic Fibers

The presently disclosed subject matter contemplates the use of synthetic fibers. Non-limiting examples of synthetic fibers suitable for use in the present disclosure include fibers made from various polymers including, by way of example and not by limitation, acrylic polymers, polyamides (including, but not limited to, Nylon 6, Nylon 6/6, Nylon 12, polyaspartic acid, polyglutamic acid), polyamines, polyimides, polyacrylics (including, but not limited to, polyacrylamide, polyacrylonitrile, esters of methacrylic acid and acrylic acid), polycarbonates (including, but not limited to, polybisphenol A carbonate, polypropylene carbonate), polydienes (including, but not limited to, polybutadiene, polyisoprene, polynorbomene), polyepoxides, polyesters (including, but not limited to, polyethylene terephthalate, polybutylene terephthalate, polytrimethylene terephthalate, polycaprolactone, polyglycolide, polylactide, polyhydroxybutyrate, polyhydroxyvalerate, polyethylene adipate, polybutylene adipate, polypropylene succinate), polyethers (including, but not limited to, polyethylene glycol (polyethylene oxide), polybutylene glycol, polypropylene oxide, polyoxymethylene (paraformaldehyde), polytetramethylene ether (polytetrahydrofuran), polyepichlorohydrin), polyfluorocarbons, formaldehyde polymers (including, but not limited to, urea-formaldehyde, melamine-formaldehyde, phenol formaldehyde), natural polymers (including, but not limited to, cellulosics, chitosans, lignins, waxes), polyolefins (including, but not limited to, polyethylene, polypropylene, polybutylene, polybutene, polyoctene), polyphenylenes (including, but not limited to, polyphenylene oxide, polyphenylene sulfide, polyphenylene ether sulfone), silicon containing polymers (including, but not limited to, polydimethyl siloxane, polycarbomethyl silane), polyurethanes, polyvinyls (including, but not limited to, polyvinyl butyral, polyvinyl alcohol, esters and ethers of polyvinyl alcohol, polyvinyl acetate, polystyrene, polymethylstyrene, polyvinyl chloride, polyvinyl pryrrolidone, polymethyl vinyl ether, polyethyl vinyl ether, polyvinyl methyl ketone), polyacetals, polyarylates, and copolymers (including, but not limited to, polyethylene-co-vinyl acetate, polyethylene-co-acrylic acid, polybutylene terephthalate-co-polyethylene terephthalate, polylauryllactam-block-polytetrahydrofuran), polybutylene succinate and polylactic acid based polymers, derivatives thereof, copolymers thereof, and the like, or combinations thereof. In certain embodiments, these polymer materials can be used in a monocomponent fiber. Alternatively, two or more polymer materials can be used together in a bicomponent fiber, e.g., a high core bicomponent fiber or a low core bicomponent fiber.

### Binders

In certain non-limiting embodiments, the fibrous substrate can include binders. Suitable binders include, but are not limited to, liquid binders and powder binders. Non-limiting examples of liquid binders include emulsions, solutions, or suspensions of binders.

Suitable binders include, but are not limited to, copolymers, including vinyl-chloride containing copolymers such as Wacker Vinnol 4500, Vinnol 4514, and Vinnol 4530, vinylacetate ethylene (VAE) copolymers, which can have a stabilizer such as Wacker Vinnapas 192, Wacker Vinnapas EF 539, Wacker Vinnapas EP907, Wacker Vinnapas EP129, Celanese Duroset E130, Celanese Dur-O-Set Elite 130 25-1813 and Celanese Dur-O-Set TX-849, Celanese 75-524A, polyvinyl alcohol-polyvinyl acetate blends such as Wacker Vinac 911, vinyl acetate homopolymers, polyvinyl amines such as BASF Luredur, acrylics, cationic acrylamides, polyacrylamides such as Bercon Berstrength 5040 and Bercon Berstrength 5150, hydroxyethyl cellulose, starch such as National Starch CATO RTM 232, National Starch CATO RTM 255, National Starch Optibond, National Starch Optipro, or National Starch OptiPLUS, guar gum, styrene-butadienes, urethanes, urethane-based binders, thermoplastic binders, acrylic binders, and carboxymethyl cellulose such as Hercules Aqualon CMC. In certain embodiments, the binder is a natural polymer-based binder. Non-limiting examples of natural polymer-based binders include polymers derived from starch, cellulose, chitin, and other polysaccharides.

In certain embodiments, the binder is water-soluble. In one embodiment, the binder is an ethylene-vinyl acetate (EVA) copolymer. One non-limiting example of such copolymers is ethylene-vinyl acetate-based Vinnapas EP907 (Wacker Chemicals, Munich, Germany). Ethylene-vinyl acetate-based Vinnapas EP907 can be applied at a level of about 10% solids incorporating about 0.75% by weight Aerosol OT (Cytec Industries, West Paterson, N.J.), which is an anionic surfactant. Other classes of liquid binders such as styrene-butadiene and acrylic binders can also be used.

In certain embodiments, the binder is salt sensitive and water-soluble. In certain embodiments, the binder is sodium CMC or calcium CMC. In certain embodiments, the binder can be temporary wet strength agents including, but not limited to Diallyldimethylammonium Chloride (DADMAC), Polydiallyldimethylammonium chloride (polyDADMAC), N-methylolacrylamide (NMA), polyacrylamide (PAM), glyoxylated polyacrylamide (GPAM), polyamide epichlorohydrin (PAE), polyamidoamine epichlorohydrin (PAAE) or combinations thereof. Other binders could be sodium CMC cross-linked with carboxylic acid, cationic ion sensitive binder, a, or a soluble starch. Any water-soluble binder that is not significantly soluble in the miscible solution with reduced polarity should increase strength, dispersibility, and shape retention.

In certain embodiments, the binder can be added to the substrate in an amount of about 1% to about 5%, about 1% to about 3%, or about 2% to about 3% by weight, on a dry basis. In particular embodiments, the binder can be added to the substrate in an amount of about 1%, about 2%, about 2.45%, about 2.5%, about 2.9%, about 3%, about 4%, or about 5% by weight, on a dry basis.

### E. Features

As embodied herein, formulations of the present disclosure are suitable for use with fibrous substrates, for example, premoistened personal care wipes. The formulations provide fibrous substrates having increased wet strength without binders, adhesives, or multiple plies of material. The formulations further provide fibrous substrates having high dispersibility that are safe to use in septic tanks, home plumbing, hospitals, and municipal water treatment sources. Thus, fibrous substrates including formulations of the present disclosure advantageously include several features such as increased wet strength, high dispersibility, shape retention, and effective cleaning properties.

### Wet Strength and Dispersibility

Dispersible fibrous substrates have two sources of strength. One is fiber-to-fiber interaction, and one is binder strength from binders.

As provided herein, fibrous substrates can include both natural cellulosic pulp fibers and man-made regenerated cellulose fibers and can be used as a substrate. Regenerated cellulose is produced by dissolving high-cellulose content wood pulp (i.e., dissolving pulp grade) in a solvent (NMMO) and extruding the dope into a coagulation bath to form continuous filaments. These filaments are then chopped into individual fibers at a given length for the desired application, for example, about 6 mm. These fibers are 100% cellulosic and biodegradable. The manufacturing process highly orients the cellulose chains along the axis of the fiber, enhancing the strength of these fibers, especially in a wetted state. Therefore, Tencel® has a higher tenacity when wet than other regenerated cellulose fibers such as rayon or viscose.

A natural cellulosic pulp fiber comprises microfibrils. As delamination of the microfibrils occurs through mechanical or chemical action, smaller microfibrils are loosened from fiber bundles and made available to entangle with other fibers and microfibrils. In a water-based web, it is primarily physical entanglement and associated physical forces that contribute to the sheet strength. More fibrillation and fiber to fiber contact will increase the available bonding area and increase the wet tensile strength. As the sheet dries, it consolidates, and capillary forces influence the van der Waal interactions that take place between the fibers. Thus, with increasing solids content, tensile strength will also increase. However, with a low dielectric solvent, hydrogen bonding can take the place of hydroentanglement as a source of tensile strength thereby allowing for wet strength and rapid dispersibility in tap water to coexist. As drying progresses (particularly under restraint or pressing) the fibers become flatter so that H-bond formation can collapse the lumen building in additional H-bonding. The collapsed fibers are ribbon-like and expose a greater area for further H-bonding between fiber surfaces.

Equation 1 is a common model used to measure the tensile strength of paper. The relationship can be simplified to the contributions from individual fiber strength and the strength of fiber-to-fiber bonding.

$$\frac{1}{Tensile strength} = \frac{1}{fiber strength} + \frac{1}{bond strength} \qquad Equation 1$$

H-bonding between fibers can only occur where there is a junction between fibers or microfibrils. To enhance the relative bonding area, it is common to utilize a mixture of long and short fibers to “fill in” the sheet and build strength through a continuous bonding network. Reactive dry strength agents (i.e., cationic starch) serve the purpose of increasing the available bonding area and thus the number of H-bonds. While the retention is based on the ionic interaction the starch and the carboxyl group on the cellulose, the strength is derived mainly from H-bonding. When wetted the H-bonding will be disrupted and the product will disperse at a rate dependent upon the molecular size and charge density (and the dielectric constant of the solvent/water mixture). Low dielectric solvents can leverage reactive dry strength agents to boost the base sheet strength without significantly damaging the hydrogen bonding of the sheet, whether the sheet is pre-moistened or post-moistened.

In addition to H-bonding, binders can be used to provide wet strength. One binder can be temporary wet strength agents including, but not limited to Diallyl dimethylammonium Chloride (DADMAC), N-methylolacrylamide (NMA), polyacrylamide (PAM), glyoxylated polyacrylamide (GPAM), polyamide epichlorohydrin (PAE), polyamidoamine epichlorohydrin (PAAE) or combinations thereof. Other binders could be sodium CMC cross-linked with carboxylic acid, cationic ion sensitive binder, or a soluble starch. Any water-soluble binder that is not significantly soluble in the miscible solution with reduced polarity should increase strength, dispersibility, and shape retention.

The use of miscible compositions to reduce the polarity of the formulations of the present disclosure minimizes the solubility of binders that would normally be highly soluble in water thereby increasing the premoistened wipe strength. In fact, when polarity is reduced enough, over 90% of the dry sheet strength of the fibrous substrate is maintained. It is further believed that miscible solvents provide strong fibrous substrates, but also for instantaneous dispersion. As the fibrous substrate is in contact with water, the miscible solvent flashes off and the fibrous substrate disperses. For water-soluble binders that use salt, salt can be used in combination with a low polarity formulation to lower the solubility of the binder further. Additionally, salt and a low polarity solvent can be balanced to produce a strong and dispersible fibrous substrate. Blended systems can also be used to provide low polarity solvents to produce and strong and dispersible fibrous substrate. Thus, the formulations of the present disclosure advantageously and unexpectedly simultaneously impart increased wet strength and high dispersibility properties to fibrous substrates. This is, the solvent formulations presently disclosed balance between delivering a fibrous substrate with increased wet strength and a high dispersibility.

In certain embodiments, fibrous substrates treated with formulations of the present disclosure can have strength while having high dispersibility. In certain embodiments, such treated fibrous substrates can have a ball burst force of from about 0.1 lb. to about 5 lb., about 0.5 lb. to about 4 lb., or from about 1 lb. to about 3 lb. In particular embodiments, such treated fibrous substrates can have a ball burst force of at least about 0.1 lb., about 0.5 lb., about 1 lb., about 1.5 lb., about 2 lb., about 2.5 lb., about 3 lb., or about 4 lb. The treated fibrous materials of the present disclosure can have a ball burst dry retention (% dry) of from about 5% to about 100%, about 10% to about 70%, or from about 15% to about 50%. The treated fibrous materials of the present disclosure can have a cross-direction (CD) tensile strength (% dry) of from about 1% to about 70%, about 5% to about 60%, or from about 10% to about 50%. The treated fibrous materials of the present disclosure can have machine direction (MD) tensile strength (% dry) of from about 1% to about 70%, about 5% to about 60%, or from about 10% to about 50%. The treated fibrous materials can have a cross-direction (CD) tensile strength (gli) of from about 1 gli to about 2,000 gli, from about 40 gli to about 1,500 gli, or from about 50 gli to about 800 gli. The treated fibrous materials can have a machine direction (MD) tensile strength (gli) of from about 1 gli to about 2,000 gli, from about 40 gli to about 1,500 gli, or from about 50 gli to about 800 gli.

### Additional Features

Formulations of the present disclosure have several additional advantages. Specifically, the presently disclosed formulations do not require toxic materials such as boric acid and provide less sticky fibrous substrates as compared to currently available commercial alternatives. Additionally, the formulations provide for increased stability compared to formulations including solvents that separate in water like isopropyl alcohol. Formulations of the present disclosure additionally eliminate malodors generated by binding systems that use amines such as lysine. Further, formulations of the present disclosure provide fibrous substrates in which dispersion effects are independent of pH. Thus, end products can be produced near human skin pH (i.e., 4.5 to 5.5) for increased consumer comfort. The formulations of the present disclosure do not require high levels of salt, and thus products can be produced with similar ionic strength of the human body for a gentler product. Additionally, formulations of the present disclosure provide for reduced foaming during perineal cleaning that is undesired by consumers. The presently disclosed formulations have another advantage in that they provide a binder agnostic for water dispersible binders. Formulations of the present disclosure provide any binder, temporary wet strength agent, or sheet strengthened by hydrogen bonding to maintain strength while simultaneously exhibiting high dispersibility. Formulations of the present disclosure further allow fibrous substrates, such as wipes, to maintain embossing patterns for improved cleaning by increasing the retention of formed or embossed patterns. Such an advantage also provides for increased brand recognition. Additionally, formulations of the present disclosure maintain wet strength of temporary wet strength (TWS) in the presence of preservatives.

### F. Applications

The presently disclosed formulations can be used in a wide variety of applications and can be suitable for use with fibrous substrates. As embodied herein, the formulations can be used for application to a fibrous substrate, such as a premoistened personal care wipe. In certain embodiments, the formulations can be used for application with consumer products such as personal care wipes, baby wipes, cosmetic wipes, toilet cleaning wipes, hard surface disinfecting wipes, bathroom cleaning wipes, kitchen cleaning wipes, feminine hygiene cleaning wipes, medicated wipes, wound cleaning wipes, perineal wipes, makeup removing wipes, hand cleaning wipes, facial cleaning and refreshing wipes, biohazard cleaning wipes, polishing wipes, mechanics hand cleaning wipes, eye glass cleaning wipes, disinfecting wipes, hand sanitizing wipes, food contact sanitizing wipes, furniture wipes, or floor cleaning wipes.

In certain embodiments, the formulations of the present disclosure are acceptable in cosmetic products. Such cosmetic products are known in the art and include but are not limited to such products identified by the Personal Care Products Council (PCPC). Typically, allowable limits of such formulations used in cosmetic products are determined by safety testing as deemed appropriate for the application.

In addition to their use on premoistened wipes, formulations of the present disclosure can be suitable for use on dry substrates, for example, as a spray on for dry materials such as dry toilet paper. Thus, the formulations of the present disclosure can further be used in spray systems as a wet spray.

The efficacy of the formulation can be observed, for example, in treating a premoistened or dry fibrous substrate with the formulation and testing for strength and dispersibility. Strength tests have demonstrated that after treatment with the formulation of the present disclosure, fibrous substrates have maintained over 90% of their dry strength. Further dispersibility tests have demonstrated that after treatment with the formulation of the present disclosure, fibrous substrates have high dispersibility properties.

The presently disclosed formulations advantageously provide a fibrous substrate having both increased wet strength and high dispersibility. Formulations of the present disclosure have a low dielectric constant which increases wet strength, decreases foaming, maintains cleaning, and maintains integrity of embossed or formed patterns of fibrous substrates.

### G. Kits

The presently disclosed formulations can be provided in one or more kits for consumer use. The kits can include, for example and not by way of limitation, one or more formulations of the present disclosure. The kits can also include one or more fibrous substrates, such as wipes. For example, the kits can include one or more of personal care wipes, baby wipes, cosmetic wipes, toilet cleaning wipes, hard surface disinfecting wipes, bathroom cleaning wipes, kitchen cleaning wipes, vaginal cleaning wipes, medicated wipes, wound cleaning wipes, perineal wipes, makeup removing wipes, hand cleaning wipes, facial cleaning and refreshing wipes, biohazard cleaning wipes, polishing wipes, mechanics hand cleaning wipes, eye glass cleaning wipes, disinfecting wipes, hand sanitizing wipes, food contact sanitizing wipes, furniture wipes, or floor cleaning wipes. In one embodiment, the kit includes a formulation solution separate from a wipe material. In an alternative embodiment, the kit can include a premoistened wipe including one or more formulations of the present disclosure. Optionally, such kits can further include any of the other systems components described in relation to the present disclosure and any other materials or items relevant to the present disclosure.

### 6. EXAMPLES

The following examples are merely illustrative of the presently disclosed subject matter and they should not be considered as limiting the scope of the subject matter in any way.

### Example 1: Solvent Effects on Hydrogen Bonding and Binder Dissolution

This Example summarizes solvent effects on hydrogen bonding and binder dissolution.

Hydrogen Bonding

Solvent effects on hydrogen bonding were tested. Several solvents (water, Zemea, propylene glycol, hexylene glycol, and mineral oil) were tested for wet tensile strength.

For tensile testing, a precision paper cutter was used to cut 1″×5″ wipe samples. The strips were placed into pneumatic grips with 1.5″×1″ facings with flat cutters and an Instron elongation tensile tester. The strips were placed into the pneumatic grips and using the Instron elongation tensile tester, and tensile forces were applied until the sheet broke. The process was repeated three times and the average recorded. The tensile strength was tested in the machine direction (MD) and the cross-machine direction (CD). The methods used herein are in part provided in ASTM Standard D5035-06 and INDA Standard Test: WSP 110.4 (05) Standard Test Method for Breaking for and Elongation of Nonwoven Materials (Strip Method), which are incorporated by reference herein in their entireties. The INDA Standard Test: WSP 110.4 (05) Standard Test Method for Breaking for and Elongation of Nonwoven Materials (Strip Method) uses a 1″ width specimen, 3 in. jaw span and 12 in/min. cross-head speed.

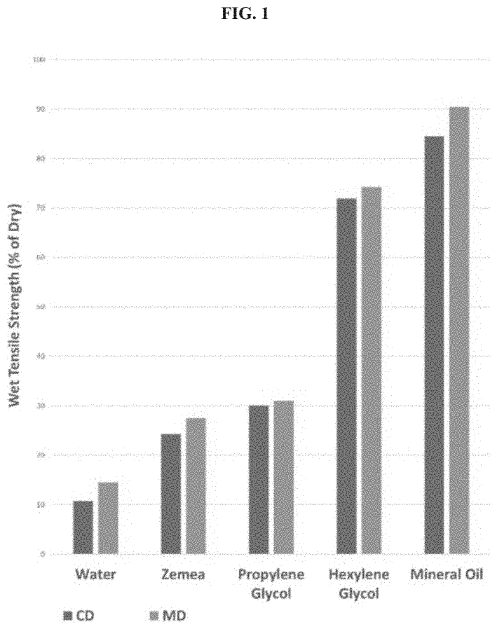

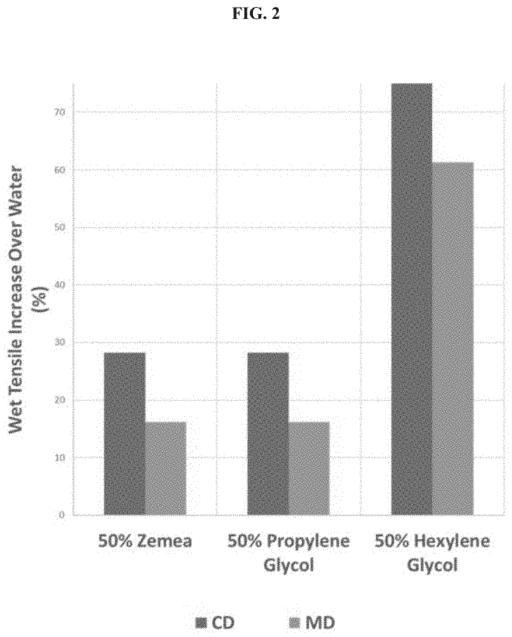

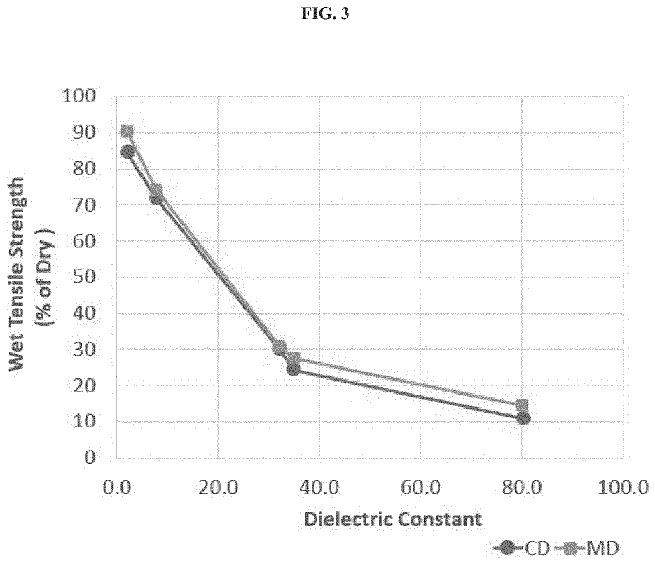

The test results are provided in FIGS. 1-3. For base sheets that depend on hydrogen bonding for strength, as shown in the premoistened tissue effects shown in FIGS. 1 and 2, reducing the dielectric constant of the solvent preserves the hydrogen bonding and wet strength of the sheet. As shown in FIG. 1, the hydrogen bonding is protected since water (DE=80.1), Zemea or 1,3 propane diol (DE=35.0), propylene glycol (DE=32), hexylene glycol (DE=7.7), and mineral oil (DE=2.1). Between the dielectric constant of 32 and 7.7 an increase in strength was observed. With dispersible base sheets used in Example 3 differences in sheet strength were observed with butylene glycol (DE=28.8), nut not with propylene glycol (DE=32) indicating that the region is an area in which water loses the ability to affect hydrogen bonding. Blends of solution with tissue base sheets provide similar results as shown in FIG. 2. FIG. 3 is a graph plotted the dielectric constant effect versus tissue tensile strength, which provided for increasing tensile strength near a dielectric constant of 30.

Binder Dissolution

Solvent effects on binder dissolution were tested. Several solvents (glycerin, isopropyl alcohol (IPA), butylene glycol, and dipropylene glycol monomethyl ether)) at varying concentrations (0 wt. %, 25 wt. %, 50 wt. %, 75 wt. %, and 100 wt. %) were tested for strength. A dry base sheet was also tested.

Dry wipes including add-ons of the solvents were tested according to INDA Wiper Ball Burst Test Method WSP 110.5 R4 (12), which is incorporated by reference herein its entirety, to test the ball burst force. The Ball Burst test used an Instron Tensile Tester and 200 lb. load cell (Instron Corporation, Canton, Mass.) with a ball burst fixture (MTS Systems Corporation, Stoughton, Mass.) and a 44.45 mm Ring Clamp Assembly (Research Dimensions, Neenah, Wis.). Samples were handled lightly from the corners and tightened into the assembly will a 1″ steel ball probe. The Instron Tensile Tester was calibrated per Instron instructions and run under standard ball burst conditions. The ball burst force was the maximum force recorded by the Instron Tensile Tester prior to break through.

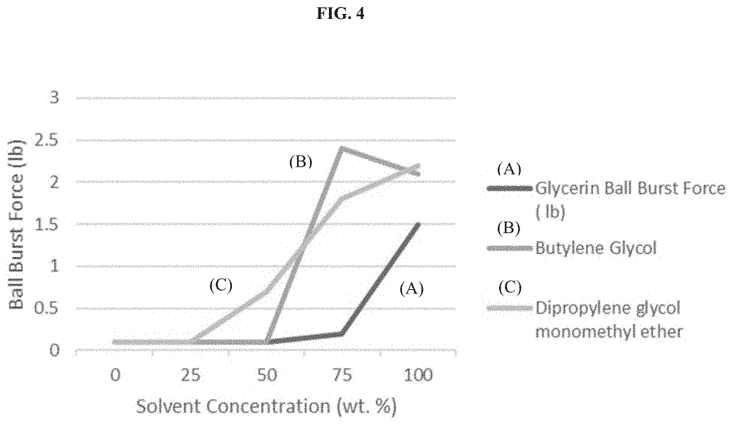

The test results are provided in FIG. 4 and Table 2.

TABLE 2

| | | Isopropyl | | Dipropylene |
|---|---|---|---|---|
| | | Alcohol | Butylene | Glycol |
| Solvent | Glycerin | (IPA) | Glycol | Monomethyl ether |
| Concentration | Ball Burst | Ball Burst | Ball Burst | Ball Burst Force |
| [wt. %] | Force [lb.] | Force [lb.] | Force [lb.] | [lb.] |
| 0 | 0.1 | 0.1 | 0.1 | 0.1 |
| 25 | 0.1 | 0.1 | 0.1 | 0.1 |
| 50 | 0.1 | 0.1 | 0.1 | 0.7 |
| 75 | 0.2 | 0.2 | 2.4 | 1.8 |
| 100 | 1.5 | 2.6 | 2.1 | 2.2 |
| Dry Ball Burst | 2.1-3.3 lb. | | | |

The materials provided self-preserving high strength (90-95% of the dry strength). Additionally, isopropyl alcohol did not show significant strength improvements at a 75% concentration due to separation in the presence of salt.

### Example 2: Solvent Modified Lotions (Lotions 1-49)

This Example summarizes various lotions for use with fibrous substrates that include water soluble binders. The lotions include a solvent that is miscible with water to reduce the dielectric constant of the lotion. The lotions can also include a coupling agent that provides phase stability when additional components such as skin protectants and/or fragrances and salts are present. Salts can be added to reduce the solubility of the binder, and thereby reduce the solvent requirement, which reduces costs, and, in some cases, safety concerns associated with high solvent loads. The chelating agent can be added only in the absence of multivalent cations. Chelating agents reduce the effects of water hardness during process and reduce the solubility of binders by reducing the amount of available binder.

The lotions were prepared with various concentrations of components as Lotions 1-49. The compositions of Lotions 1-49 are provided in Table 3 below. Each component is identified by chemical name, and where applicable, with its respective wt. % based on the overall weight of the formulation.

TABLE 3
Lotions

| | | 1 | 2 | 3 | 4 | 5 | 6 | 7 | 8 | 9 | 10 | 11 | |
|---|---|---|---|---|---|---|---|---|---|---|---|---|---|
| Component | | [wt. %] | [wt. %] | [wt. %] | [wt. %] | [wt. %] | [wt. %] | [wt. %] | [wt. %] | [wt. %] | [wt. %] | [wt. %] | |
| Deionized Water | Solvent | Q.S. | Q.S. | Q.S. | Q.S. | Q.S. | Q.S. | Q.S. | Q.S. | Q.S. | Q.S. | Q.S. | |
| Propylene Glycol | Solvent DEC | 50.00 | 50.00 | 50.00 | 50.00 | — | — | — | — | — | — | — | |
| | Reducer | | | | | | | | | | | | |
| Butylene Glycol | Solvent DEC | — | — | — | — | 50.00 | 50.00 | 50.00 | 50.00 | 50.00 | 50.00 | 50.00 | |
| | Reducer | | | | | | | | | | | | |
| Hexylene Glycol | Solvent DEC | — | — | — | — | — | — | — | — | — | — | — | |
| | Reducer | | | | | | | | | | | | |
| Glycerin | Solvent DEC | — | 10.00 | 20.00 | 30.00 | — | 10.00 | 20.00 | 30.00 | 10.00 | 10.00 | 10.00 | |
| | Reducer | | | | | | | | | | | | |
| Dipropylene Glycol | Solvent DEC | — | — | — | — | — | — | — | — | — | — | — | |
| Monomethyl Ether | Reducer | | | | | | | | | | | | |
| (Dowanol ™ DPM) | | | | | | | | | | | | | |
| CaCl₂ | | — | — | — | — | — | — | — | — | — | — | — | |
| CaCl₂•2H₂O | Sheet | — | — | — | — | — | — | — | — | — | — | — | |
| | Strengthening | | | | | | | | | | | | |
| | Agent | | | | | | | | | | | | |
| Polysorbate 20 | Coupling | 0.50 | 0.50 | 0.50 | 0.50 | 0.50 | 0.50 | 0.50 | 0.50 | 0.50 | 0.50 | 0.50 | |
| | Agent | | | | | | | | | | | | |
| Cola ® Lipid C | Skin | 1.50 | 1.50 | 1.50 | 1.50 | 1.50 | 1.50 | 1.50 | 1.50 | 1.50 | 1.50 | 1.50 | |
| | Protectant | | | | | | | | | | | | |
| Ethylenedi- | Chelating | 0.10 | 0.10 | 0.10 | 0.10 | 0.10 | 0.10 | 0.10 | 0.10 | 0.10 | 0.10 | 0.10 | |
| aminetraacetic | Agent | | | | | | | | | | | | |
| acid (EDTA) | | | | | | | | | | | | | |
| Microcare ® BDP | Preservative/ | 0.50 | 0.50 | 0.50 | 0.50 | 0.50 | 0.50 | 0.50 | 0.50 | 0.50 | 0.50 | 0.50 | |
| | DEC | | | | | | | | | | | | |
| | Adjuster | | | | | | | | | | | | |
| Fragrance | | 0.05 | 0.05 | 0.05 | 0.05 | 0.05 | 0.05 | 0.05 | 0.05 | 0.05 | 0.05 | 0.05 | |
| Sodium Chloride | | — | — | — | — | — | — | — | — | 1.00 | 2.00 | 4.00 | |
| (NaCl) | | | | | | | | | | | | | |
| | | 12 | 13 | 14 | 15 | 16 | 17 | 18 | 19 | 20 | 21 | 22 | 23 |
| Component | | [wt. %] | [wt. %] | [wt. %] | [wt. %] | [wt. %] | [wt. %] | [wt. %] | [wt. %] | [wt. %] | [wt. %] | [wt. %] | [wt. %] |
| Deionized Water | Solvent | Q.S. | Q.S. | Q.S. | Q.S. | Q.S. | Q.S. | Q.S. | Q.S. | Q.S. | Q.S. | Q.S. | Q.S. |
| Propylene Glycol | Solvent DEC | — | — | — | — | — | — | — | — | — | — | — | — |
| | Reducer | | | | | | | | | | | | |
| Butylene Glycol | Solvent DEC | 50.00 | 50.00 | 50.00 | 50.00 | 50.00 | 50.00 | 50.00 | 50.00 | 50.00 | 50.00 | 50.00 | 50.00 |
| | Reducer | | | | | | | | | | | | |
| Hexylene Glycol | Solvent DEC | — | — | — | — | — | — | — | — | — | — | — | — |
| | Reducer | | | | | | | | | | | | |
| Glycerin | Solvent DEC | 10.00 | 10.00 | 10.00 | 10.00 | 10.00 | — | — | — | — | — | — | — |
| | Reducer | | | | | | | | | | | | |
| Dipropylene Glycol | Solvent DEC | — | — | — | — | — | — | 15.00 | — | — | 15.00 | 15.00 | 15.00 |
| Monomethyl Ether | Reducer | | | | | | | | | | | | |
| (Dowanol ™ DPM) | | | | | | | | | | | | | |
| CaCl₂ | | — | 0.5 | 1.00 | 2.00 | 4.00 | — | — | — | — | — | — | — |
| CaCl₂•2H₂O | Sheet | — | — | — | — | — | — | — | 0.50 | 0.50 | 0.50 | — | 0.50 |
| | Strengthening | | | | | | | | | | | | |
| | Agent | | | | | | | | | | | | |
| Polysorbate 20 | Coupling | 0.50 | 0.50 | 0.50 | 0.50 | 0.50 | 0.50 | 0.50 | 0.50 | 0.50 | 0.50 | 0.50 | 0.50 |
| | Agent | | | | | | | | | | | | |
| Cola Lipid C | Skin | 1.50 | 1.50 | 1.50 | 1.50 | 1.50 | 1.50 | 1.50 | 1.50 | 1.50 | 1.50 | 1.50 | 1.50 |
| | Protectant | | | | | | | | | | | | |
| Ethylenedi- | Chelating | — | — | — | — | — | — | — | — | — | — | — | — |
| aminetraacetic | Agent | | | | | | | | | | | | |
| acid (EDTA) | | | | | | | | | | | | | |
| Microcare ® BDP | Preservative/ | 0.50 | 0.50 | 0.50 | 0.50 | 0.50 | 0.50 | 0.50 | 0.50 | 0.50 | 0.50 | 0.50 | 0.50 |
| | DEC | | | | | | | | | | | | |
| | Adjuster | | | | | | | | | | | | |
| Fragrance | | 0.05 | 0.05 | 0.05 | 0.05 | 0.05 | 0.05 | 0.05 | 0.05 | 0.05 | 0.05 | 0.05 | 0.05 |
| Sodium Chloride | | — | — | — | — | — | — | — | — | 1.50 | — | 1.50 | 1.50 |
| (NaCl) | | | | | | | | | | | | | |
| | | 24 | 25 | 26 | 27 | 28 | 29 | 30 | 31 | 32 | 33 | 34 | 35 |
| Component | | [wt. %] | [wt. %] | [wt. %] | [wt. %] | [wt. %] | [wt. %] | [wt. %] | [wt. %] | [wt. %] | [wt. %] | [wt. %] | [wt. %] |
| Deionized Water | Solvent | Q.S. | Q.S. | Q.S. | Q.S. | Q.S. | Q.S. | Q.S. | Q.S. | Q.S. | Q.S. | Q.S. | Q.S. |
| Propylene Glycol | Solvent DEC | — | — | — | — | — | — | — | — | — | — | — | — |
| | Reducer | | | | | | | | | | | | |
| Butylene Glycol | Solvent DEC | 50.00 | 50.00 | 50.00 | 50.00 | 50.00 | 50.00 | 50.00 | 50.00 | 50.00 | 50.00 | 50.00 | 50.00 |
| | Reducer | | | | | | | | | | | | |
| Hexylene Glycol | Solvent DEC | — | 5.00 | 5.00 | 5.00 | 5.00 | 5.00 | 5.00 | 5.00 | 5.00 | 5.00 | 10.00 | 10.00 |
| | Reducer | | | | | | | | | | | | |
| Glycerin | Solvent DEC | — | — | — | — | — | — | — | — | — | — | — | — |
| | Reducer | | | | | | | | | | | | |
| Dipropylene Glycol | Solvent DEC | — | — | — | — | — | — | — | — | — | — | — | — |
| Monomethyl Ether | Reducer | | | | | | | | | | | | |
| (Dowanol ™ DPM) | | | | | | | | | | | | | |
| CaCl₂ | | — | — | — | — | 0.50 | 1.00 | 1.50 | 2.00 | 2.50 | 4.00 | — | — |
| CaCl₂•2H₂O | Sheet | — | — | — | 0.50 | — | — | — | — | — | — | — | — |
| | Strengthening | | | | | | | | | | | | |
| | Agent | | | | | | | | | | | | |
| Polysorbate 20 | Coupling | 0.50 | 0.50 | 0.50 | 0.50 | 0.50 | 0.50 | 0.50 | 0.50 | 0.50 | 0.50 | 0.50 | 0.50 |
| | Agent | | | | | | | | | | | | |
| Cola Lipid C | Skin | 1.50 | 1.50 | 1.50 | 1.50 | 1.50 | 1.50 | 1.50 | 1.50 | 1.50 | 1.50 | 1.50 | 1.50 |
| | Protectant | | | | | | | | | | | | |
| Ethylenedi- | Chelating | — | — | — | — | — | — | — | — | — | — | — | — |
| aminetraacetic | Agent | | | | | | | | | | | | |
| acid (EDTA) | | | | | | | | | | | | | |
| Microcare ® BDP | Preservative/ | 0.50 | 0.50 | 0.50 | 0.50 | 0.50 | 0.50 | 0.50 | 0.50 | 0.50 | 0.50 | 0.50 | 0.50 |
| | DEC | | | | | | | | | | | | |
| | Adjuster | | | | | | | | | | | | |
| Fragrance | | 0.05 | 0.05 | 0.05 | 0.05 | 0.05 | 0.05 | 0.05 | 0.05 | 0.05 | 0.05 | 0.05 | 0.05 |
| Sodium Chloride | | — | — | 1.50 | — | — | — | — | — | — | — | — | 1.50 |
| (NaCl) | | | | | | | | | | | | | |

| | | 36 | 37 | 38 | 39 | 40 | 41 | 42 | 43 | 44 | 45 | 46 |
|---|---|---|---|---|---|---|---|---|---|---|---|---|
| Component | | [wt. %] | [wt. %] | [wt. %] | [wt. %] | [wt. %] | [wt. %] | [wt. %] | [wt. %] | [wt. %] | [wt. %] | [wt. %] |
| Deionized Water | Solvent | Q.S. | Q.S. | Q.S. | Q.S. | Q.S. | Q.S. | Q.S. | Q.S. | Q.S. | Q.S. | Q.S. |
| Propylene Glycol | Solvent DEC | — | — | — | — | — | — | — | — | — | — | — |
| | Reducer | | | | | | | | | | | |
| Butylene Glycol | Solvent DEC | 50.00 | 50.00 | 50.00 | 50.00 | Q.S. | 20.00 | 30.00 | 40.00 | 50.00 | 30.00 | 30.00 |
| | Reducer | | | | | | | | | | | |
| Hexylene Glycol | Solvent DEC | 10.00 | 10.00 | 10.00 | 10.00 | 5.00 | 5.00 | 5.00 | 5.00 | 5.00 | 5.00 | 5.00 |
| | Reducer | | | | | | | | | | | |
| Glycerin | Solvent DEC | — | — | — | — | — | — | — | — | — | — | — |
| | Reducer | | | | | | | | | | | |
| Dipropylene Glycol | Solvent DEC | — | — | — | — | — | — | — | — | — | | |
| Monomethyl Ether | Reducer | | | | | | | | | | | |
| (Dowanol ™ DPM) | | | | | | | | | | | | |
| CaCl₂ | | — | 0.50 | 1.00 | 1.50 | — | — | — | — | — | — | 0.37 |
| CaCl₂•2H₂O | Sheet | 0.50 | — | — | — | 2.50 | 2.50 | 2.50 | 2.50 | 2.50 | Q.S. | Q.S. |
| | Strengthening | | | | | | | | | | | |
| | Agent | | | | | | | | | | | |
| Polysorbate 20 | Coupling | 0.50 | 0.50 | 0.50 | 0.50 | 0.50 | 0.50 | 0.50 | 0.50 | 0.50 | 0.50 | 0.50 |
| | Agent | | | | | | | | | | | |
| Cola Lipid C | Skin | 1.50 | 1.50 | 1.50 | 1.50 | — | — | — | — | — | — | — |
| | Protectant | | | | | | | | | | | |
| Ethylenedi- | Chelating | — | — | — | — | — | — | — | — | — | — | — |
| aminetraacetic | Agent | | | | | | | | | | | |
| acid (EDTA) | | | | | | | | | | | | |
| Microcare ® BDP | Preservative/ | 0.50 | 0.50 | 0.50 | 0.50 | 0.50 | 0.50 | 0.50 | 0.50 | 0.50 | 0.50 | 0.50 |
| | DEC | | | | | | | | | | | |
| | Adjuster | | | | | | | | | | | |
| Fragrance | | 0.05 | 0.05 | 0.05 | 0.05 | 0.05 | 0.05 | 0.05 | 0.05 | 0.05 | 0.05 | 0.05 |
| Sodium Chloride | | — | — | — | — | — | — | — | — | — | — | — |
| (NaCl) | | | | | | | | | | | | |

| | | 47 | 48 | 49 |
|---|---|---|---|---|
| Component | | [wt. %] | [wt. %] | [wt. %] |
| Deionized Water | Solvent | Q.S. | Q.S. | Q.S. |
| Propylene Glycol | Solvent DEC | — | — | — |
| | Reducer | | | |
| Butylene Glycol | Solvent DEC | 30.00 | 30.00 | 30.00 |
| | Reducer | | | |
| Hexylene Glycol | Solvent DEC | 5.00 | 5.00 | 5.00 |
| | Reducer | | | |
| Glycerin | Solvent DEC | — | — | — |
| | Reducer | | | |
| Dipropylene Glycol | Solvent DEC | — | — | — |
| Monomethyl Ether | Reducer | | | |
| (Dowanol ™ DPM) | | | | |
| CaCl₂ | | 0.90 | 1.70 | 2.50 |
| CaCl₂•2H₂O | Sheet | Q.S. | Q.S. | Q.S. |
| | Strengthening | | | |
| | Agent | | | |
| Polysorbate 20 | Coupling | 0.50 | 0.50 | 0.50 |
| | Agent | | | |
| Cola Lipid C | Skin | — | — | — |
| | Protectant | | | |
| Ethylenedi- | Chelating | — | — | — |
| aminetraacetic | Agent | | | |
| acid (EDTA) | | | | |
| Microcare ® BDP | Preservative/ | 0.50 | 0.50 | 0.50 |
| | DEC | | | |
| | Adjuster | | | |
| Fragrance | | 0.05 | 0.05 | 0.05 |
| Sodium Chloride | | — | — | — |
| (NaCl) | | | | |

### Example 3: Strength Testing (Lotions 1-49)

This Example tested the strength of fibrous substrates including lotions disclosed in Example 2, as provided below. The lotions were applied to a dried wipe as an add-on. An add-on of 200% or 250% was applied. For example, a 200% add-on indicates that the weight of the applied lotion is 2× the weight of the dry wipe.

The dry wipes including add-ons of the lotions were tested according to INDA Wiper Ball Burst Test Method WSP 110.5 R4 (12), which is incorporated by reference herein its entirety, to test the ball burst force. The Ball Burst test used an Instron Tensile Tester and 200 lb. load cell (Instron Corporation, Canton, Mass.) with a ball burst fixture (MTS Systems Corporation, Stoughton, Mass.) and a 44.45 mm Ring Clamp Assembly (Research Dimensions, Neenah, Wis.). Samples were handled lightly from the corners and tightened into the assembly will a 1″ steel ball probe. The Instron Tensile Tester was calibrated per Instron instructions and run under standard ball burst conditions. The ball burst force was the maximum force recorded by the Instron Tensile Tester prior to break through.

Propylene Glycol and Glycerin (Lotions 1-4)

Lotions 1-4 were tested for strength. Lotions 1-4 included propylene glycol (50 wt. %), glycerin at varying weight percentages, and other additional components. The lotions were tested with a 200% add-on and with a 250% add-on. Each lotion was tested twice. The test results are provided in Table 4.

TABLE 4

| Lotion | Glycerin [wt. %] | Add-on [%] | Ball Burst [lb.] |
|---|---|---|---|
| 1 | — | 200 | 0.1 |
| 2 | 10.00 | 200 | 0.1 |
| 3 | 20.00 | 200 | 0.1 |
| 4 | 30.00 | 200 | 0.15 |
| 1 | — | 250 | 0.1 |
| 2 | 10.00 | 250 | 0.1 |
| 3 | 20.00 | 250 | 0.1 |
| 4 | 30.00 | 250 | 0.2 |

Butylene Glycol and Glycerin (Lotions 5-8)

Lotions 5-8 were tested for strength. Lotions 5-8 included butylene glycol (50 wt. %), glycerin at varying weight percentages, and other additional components. The lotions were tested with a 200% add-on and with a 250% add-on. Each lotion and add-on combination was tested twice. The test results are provided in Table 5.

TABLE 5

| Lotion | Glycerin [wt. %] | Add-on [%] | Ball Burst [lb.] |
|---|---|---|---|
| 5 | — | 250 | 0.1 |
| 5 | — | 250 | 0.1 |
| 6 | 10.0 | 250 | 0.1 |
| 6 | 10.0 | 250 | 0.1 |
| 7 | 20.0 | 250 | 0.1 |
| 7 | 20.0 | 250 | 0.1 |
| 8 | 30.0 | 250 | 0.1 |
| 8 | 30.0 | 250 | 0.2 |
| 5 | — | 200 | 0.1 |
| 5 | — | 200 | 0.1 |
| 6 | 10.0 | 200 | 0.1 |
| 6 | 10.0 | 200 | 0.1 |
| 7 | 20.0 | 200 | 0.1 |
| 7 | 20.0 | 200 | 0.1 |
| 8 | 30.0 | 200 | 0.2 |
| 8 | 30.0 | 200 | 0.2 |

Sodium Chloride (Lotions 6 and 9-11)

Lotions 6 and 9-11 were tested for strength. Lotions 9-11 included butylene glycol (50 wt. %), glycerin (10 wt. %), sodium chloride at varying weight percentages, and other additional components. The lotions were tested with a 250% add-on. Each lotion was tested twice. The test results are provided in Table 6.

TABLE 6

| Lotion | NaCl [wt. %] | Add-on [%] | Ball Burst [lb.] |
|---|---|---|---|
| 6 | — | 250 | 0.1 |
| 6 | — | 250 | 0.1 |
| 9 | 1.00 | 250 | 1.0 |
| 9 | 1.00 | 250 | 1.1 |
| 10 | 2.00 | 250 | 1.1 |
| 10 | 2.00 | 250 | 1.2 |
| 11 | 4.00 | 250 | 1.1 |
| 11 | 4.00 | 250 | 1.2 |

Calcium Chloride (Lotions 12-16)

Lotions 12-16 were tested for strength. Lotions 12-16 included butylene glycol (50 wt. %), glycerin (10 wt. %), calcium chloride at varying weight percentages, and other additional components. Ethylenediaminetetraacetic acid (EDTA) was removed to allow the effects of calcium chloride to impact the lotions. The lotions were tested with a 250% add-on. Each lotion was tested twice. The test results are provided in Table 7.

TABLE 7

| | CaCl₂•2H₂O | | |
|---|---|---|---|
| Lotion | [wt. %] | Add-on [%] | Ball Burst [lb.] |
| 12 | — | 250 | 0.1 |
| 12 | — | 250 | 0.1 |
| 13 | 0.50 | 250 | 1.5 |
| 13 | 0.50 | 250 | 1.6 |
| 14 | 1.00 | 250 | 1.6 |
| 14 | 1.00 | 250 | 1.6 |
| 15 | 2.00 | 250 | 1.5 |
| 15 | 2.00 | 250 | 1.4 |
| 16 | 4.00 | 250 | 1.7 |
| 16 | 4.00 | 250 | 1.8 |

Calcium chloride provided for increased strength as exemplified in Lotions 13-16. Lotion 16 had a ball burst of 1.8 lb. The increase in the ball burst is believed to be caused by reduced solubility of calcium CMC in the presence of calcium chloride and solvent. Because the dielectric constant of the lotion is lower, the effects of calcium chloride are greater than in a pure water system.

Dipropylene Glycol Monomethyl Ether (Dowanol DMP) and Salts (Lotions 18-23)

Lotions 18-23 were tested for strength. Lotions 18-23 included butylene glycol (50 wt. %), varying components of sodium chloride, Dowanol DPM, and calcium chloride dihydrate, and other additional components. The lotions were tested with a 250% add-on. The test results are provided in Table 8.

TABLE 8

| | | | Dowanol | | Average |
|---|---|---|---|---|---|
| | CaCl2•2H2O | NaCl | DPM | Add-on | Ball Burst |
| Lotion | [wt. %] | [wt. %] | [wt. %] | [%] | [lb.] |
| 18 | — | — | 15.00 | 250 | 0.7 |
| 19 | 0.50 | — | — | 250 | 1.3 |
| 20 | 0.50 | 1.50 | — | 250 | 1.1 |
| 21 | 0.50 | — | 15.00 | 250 | 1.9 |
| 22 | — | 1.50 | 15.00 | 250 | 1.7 |
| 23 | 0.50 | 1.50 | 15.00 | 250 | 2.1 |

By replacing glycerin with Dowanol DMP, lotions suitable for use as personal care lotions and hard surface cleaning lotions were evaluated. Dowanol DMP can be suitable for both personal care and cleaning lotion applications, though it does have a pungent odor that can limit personal care applications. Glycol ethers can be suitable cleaners and builders like sodium carbonate and can be further added to lotions of the present disclosure to increase strength and cleaning properties of fibrous substrates. The dielectric constant of Dowanol DPM (DEC=10.5) is lower than glycerin (DEC=46.5).

Lotion 18 with 15 wt. % Dowanol DPM provided 0.7 lb. ball bust force. Lotion 8 with 30 wt. % glycerin provided a 0.2 lb. ball bust force as provided in Table 5. Dowanol DPM provided an increase in sheet strength. Sheet-strengthening was also provided by calcium chloride dihydrate. The results of Lotion 23 are almost double the results for Lotion 20. Lotion 5 with 50 wt. % butylene glycol alone provided a 0.1 lb. ball bust force as provided in Table 5. Thus, solvent modification doubled the effects of salt.

Hexylene Glycol (Lotions 25-39)

Lotions 25-29 were tested for strength. Lotions 25-39 included butylene glycol (50 wt. %), hexylene glycol, varying weight percentages of salts (sodium chloride, calcium chloride, and calcium chloride dihydrate), and other additional components. Hexylene glycol is nearly odorless and has a dielectric constant (DEC=7.7) lower than Dowanol DPM (DEC=10.5). The lotions were tested with a 250% add-on. The test results are provided in Table 9.

TABLE 9

| | Hexylene | | | | | Ball |
|---|---|---|---|---|---|---|
| | Glycol | CaCl₂ | CaCl₂•2H2O | NaCl | Add-on | Burst |
| Lotion | [wt. %] | [wt. %] | [wt. %] | [wt. %] | [%] | [lb.] |
| 25 | 5.00 | — | — | — | 250 | 0.2 |
| 26 | 5.00 | — | — | 1.5 | 250 | 1.2 |
| 27 | 5.00 | — | 0.50 | — | 250 | 1.5 |
| 28 | 5.00 | 0.50 | — | — | 250 | 1.5 |
| 29 | 5.00 | 1.00 | — | — | 250 | 1.3 |
| 30 | 5.00 | 1.50 | — | — | 250 | 1.5 |
| 31 | 5.00 | 2.00 | — | — | 250 | 1.6 |
| 32 | 5.00 | 2.50 | — | — | 250 | 2.0 |
| 33 | 5.00 | 4.00 | — | — | 250 | 1.8 |
| 34 | 10.00 | — | — | — | 250 | 0.40 |
| 35 | 10.00 | — | — | 1.5 | 250 | 1.3 |
| 36 | 10.00 | — | 0.50 | — | 250 | 1.8 |
| 37 | 10.00 | 0.50 | — | — | 250 | 1.8 |
| 38 | 10.00 | 1.0 | — | — | 250 | 1.9 |
| 39 | 10.00 | 1.5 | — | — | 250 | 1.9 |

Butylene Glycol (Lotions 41-44)

Lotions 41-44 were tested for strength. In these tests, new base sheet samples were evaluated with the dry ball burst of 2.1 lb. Lotions 41-44 included butylene glycol at varying weight percentages, hexylene glycol (5.00 wt. %), calcium chloride dihydrate (2.50 wt. %), and other additional components. The lotions were tested with a 250% add-on. A dry base sheet was also tested. The results are provided in Table 10.

TABLE 10

| | Butylene | Hexylene | | | Ball |
|---|---|---|---|---|---|
| | Glycol | Glycol | CaCl2•2H2O | Add-on | Burst |
| Lotion | [wt. %] | [wt. %] | [wt. %] | [%] | [lb.] |
| Dry Base Sheet | — | — | — | — | 2.1 |
| 41 | 20.00 | 5.00 | 2.50 | 250 | 0.5 |
| 42 | 30.00 | 5.00 | 2.50 | 250 | 0.95 |
| 43 | 40.00 | 5.00 | 2.50 | 250 | 1.3 |
| 44 | 50.00 | 5.00 | 2.50 | 250 | 1.8 |

Lotion 42 having 5% hexylene glycol and 30% butylene glycol and 2.5% CaCl₂ exceeded 1 lb. ball burst, and exceeded 45% of the dry sheet strength, which are important strength parameters for commercially acceptable premoistened personal care wipes.

Salts—Calcium Chloride (Lotions 46-49)

Lotions 46-49 were tested for strength. Lotions 46-49 included butylene glycol (30 wt. %), hexylene glycol (5 wt. %), calcium chloride at varying weight percentages, and additional components. The lotions were tested with a 250% add-on. In these tests, new base sheet samples were evaluated with the dry ball burst of 1.96 lb. The results are provided in Table 11.

TABLE 11

| | Butylene | Hexylene | | | Ball |
|---|---|---|---|---|---|
| | Glycol | Glycol | CaCl₂ | Add-on | Burst |
| Lotion | [wt. %] | [wt. %] | [wt. %] | [%] | [lb.] |
| Dry Base Sheet | — | — | — | — | 1.96 |
| 46 | 30.00 | 5.00 | 0.37 | 250 | 0.4 |
| 47 | 30.00 | 5.00 | 0.9 | 250 | 0.7 |
| 48 | 30.00 | 5.00 | 1.7 | 250 | 0.75 |
| 49 | 30.00 | 5.00 | 2.5 | 250 | 0.75 |

Although the ball burst results were lower, the lotions maintained up to 38% of the dry ball burst strengths. Additionally, the substrate consumed 54% of the calcium, which was determined by applying Lotion 41 to the base sheet at a 250% add-on. The substrate rested for about 24 hours and the lotion was squeezed off the substrate. The reduction in calcium demonstrates a replacement of the sodium in sodium CMC with calcium. Calcium CMC is less soluble in water than sodium CMC, and even less soluble in a lotion that has a reduced dielectric constant.

### Example 4: Dispersibility Testing (Lotions 1-49)

This Examples tested lotions on wipe materials for dispersibility.

Lotions 1-49 were tested on wipe materials for dispersibility. A wipe was placed in a mason jar half full of water and shook with one gentle flick of the wrist to see if the wipes including the lotions broke apart. All tested wipes provided instant dispersibility.

For Lotions 12-16, a formal Slosh Box Test was conducted, which provided 100% wipe break up in 30 seconds. As a result, calcium chloride is indicated as providing increased strength and dispersibility.

### Example 5: Skin Feel Evaluation (Lotions 18-23)

Lotions 18-23 from Example 2, Table 3 were further tested in a skin feel evaluation. Lotions 18-23 were observed by laboratory hand feel and use as a perineal wipe. The test results are provided in Table 12.

TABLE 12

| | | | Dowanol | Add- | |
|---|---|---|---|---|---|
| | CaCl2•2H2O | NaCl | DPM | on | Skin Feel |
| Lotion | [wt. %] | [wt. %] | [wt. %] | [%] | Comments |
| 18 | — | — | 15.00 | 250 | Slick & slippery |
| | | | | | skin feel |
| 19 | 0.50 | — | — | 250 | Slick & slippery |
| | | | | | skin feel |
| 20 | 0.50 | 1.50 | — | 250 | Greasy skin feel |
| 21 | 0.50 | — | 15.00 | 250 | Greasy skin feel |
| 22 | — | 1.50 | 15.00 | 250 | Greasy skin feel |
| 23 | 0.50 | 1.50 | 15.00 | 250 | Greasy skin feel |

Modification of the dielectric constant provided for wipes being less grippy than water-based wipes, but not oil-based wipes. The lack of adhesion to the skin was observed with a reduced dielectric constant.

### Example 6: Lotioned Wipe Compositions (Samples 1-16)

This Example summarizes various lotions with water soluble binders for use with fibrous substrates and pre-moistened wipe compositions including the same. All lotions included the base formulation provided in Table 13.

TABLE 13

| Component | | Solids (wt-%) |
|---|---|---|
| Deionized Water | Solvent | Q.S. |
| CaCl₂ | | Variable |
| Butylene Glycol | Solvent DEC Reducer | Variable |
| Hexylene Glycol | Solvent DEC Reducer | 5.00 |
| Polysorbate 20 | Coupling Agent | 0.50 |
| Phenoxy Ethanol | Preservative | 0.80 |
| Fragrance | | 0.05 |
| Total | | 100 |
| pH | | 5.20 |

The lotion and wipe compositions were modified with the variables provided in Table 14. Levels of calcium chloride, butylene glycol, and carboxymethyl cellulose (CMC) varied across samples.

TABLE 14

| Variable | Levels (wt-%) | |
|---|---|---|
| CaCl₂ | 0.5, 1.5 & 2.0 | Added as ingredient to |
| | | lotion. |
| Butylene Glycol | 15, 30, & 45 | Added as ingredient to |
| | | lotion. |
| Carboxymethyl Cellulose | 2.00, 2.45 & 2.9 | Added as ingredient to |
| (CMC) | | dry basesheet. Weight |
| | | percent of CMC is on a |
| | | dry basis. |

The lotion and wipe compositions were modified with the variables provided in Table 14 for Samples 1-16 as provided in Table 15. The lotions were prepared with various concentrations of components as Lotions 50-65 all with the base formulation of Table 13.

TABLE 15

| | | Calcium | Carboxymethyl |
|---|---|---|---|
| | Butylene | Chloride | cellulose (CMC) |
| Sample | Glycol | (wt. %) | (wt. %) |
| 1 | 45 | 1.5 | 2.45 |
| (Lotion 50) | | | |
| 2 | 15 | 2.5 | 2.90 |
| (Lotion 51) | | | |
| 3 | 45 | 0.5 | 2.90 |
| (Lotion 52) | | | |
| 4 | 30 | 1.5 | 2.45 |
| (Lotion 53) | | | |
| 5 | 45 | 0.5 | 2.00 |
| (Lotion 54) | | | |
| 6 | 30 | 1.5 | 2.45 |
| (Lotion 55) | | | |
| 7 | 15 | 0.5 | 2.90 |
| (Lotion 56) | | | |
| 8 | 15 | 1.5 | 2.45 |
| (Lotion 57) | | | |
| 9 | 30 | 1.5 | 2.90 |
| (Lotion 58) | | | |
| 10 | 45 | 2.5 | 2.90 |
| (Lotion 59) | | | |
| 11 | 30 | 0.5 | 2.45 |
| (Lotion 60) | | | |
| 12 | 45 | 2.5 | 2.00 |
| (Lotion 61) | | | |
| 13 | 15 | 2.5 | 2.00 |
| (Lotion 62) | | | |
| 14 | 15 | 0.5 | 2.00 |
| (Lotion 63) | | | |
| 15 | 30 | 2.5 | 2.45 |
| (Lotion 64) | | | |
| 16 | 30 | 1.5 | 2.00 |
| (Lotion 65) | | | |

The complete compositions of Lotions 50-65 are provided in Table 16. The total for each lotion was 100 wt % solids and the pH for each lotion was 5.20.

TABLE 16

| | 50 | 51 | 52 | 53 | 54 | 55 | 56 | 57 |
|---|---|---|---|---|---|---|---|---|
| Component | [wt. %] | [wt. %] | [wt. %] | [wt. %] | [wt. %] | [wt. %] | [wt. %] | [wt. %] |
| Deionized Water | Q.S. | Q.S. | Q.S. | Q.S. | Q.S. | Q.S. | Q.S. | Q.S. |
| CaCl₂ | 1.5 | 2.5 | 0.5 | 1.5 | 0.5 | 1.5 | 0.5 | 1.5 |
| Butylene Glycol | 45.00 | 15.00 | 45.00 | 30.00 | 45.00 | 30.00 | 15.00 | 15.00 |
| Hexylene Glycol | 5.00 | 5.00 | 5.00 | 5.00 | 5.00 | 5.00 | 5.00 | 5.00 |
| Polysorbate 20 | 0.50 | 0.50 | 0.50 | 0.50 | 0.50 | 0.50 | 0.50 | 0.50 |
| Phenoxy Ethanol | 0.80 | 0.80 | 0.80 | 0.80 | 0.80 | 0.80 | 0.80 | 0.80 |
| Fragrance | 0.05 | 0.05 | 0.05 | 0.05 | 0.05 | 0.05 | 0.05 | 0.05 |
| | 58 | 59 | 60 | 61 | 62 | 63 | 64 | 65 |
| Component | [wt. %] | [wt. %] | [wt. %] | [wt. %] | [wt. %] | [wt. %] | [wt. %] | [wt. %] |
| Deionized Water | Q.S. | Q.S. | Q.S. | Q.S. | Q.S. | Q.S. | Q.S. | Q.S. |
| CaCl₂ | 1.5 | 2.5 | 0.5 | 2.5 | 2.5 | 0.5 | 2.5 | 1.5 |
| Butylene Glycol | 30.00 | 45.00 | 30.00 | 45.00 | 15.00 | 15.00 | 30.00 | 30.00 |
| Hevlene Glycol | 5.00 | 5.00 | 5.00 | 5.00 | 5.00 | 5.00 | 5.00 | 5.00 |
| Polysorbate 20 | 0.50 | 0.50 | 0.50 | 0.50 | 0.50 | 0.50 | 0.50 | 0.50 |
| Phenoxy Ethanol | 0.80 | 0.80 | 0.80 | 0.80 | 0.80 | 0.80 | 0.80 | 0.80 |
| Fragrance | 0.05 | 0.05 | 0.05 | 0.05 | 0.05 | 0.05 | 0.05 | 0.05 |

Lotions 50-65 were formulated were using the components listed in the base formulation of Table 13 with varying levels of butylene glycol and calcium chloride provided in Table 15, the total compositions of which are provided in Table 16. The lotions were each added to a basesheet at a 200% add-on level, i.e., the weight of the lotion added was twice the weight of the dry basesheet. The lotions were applied using an aerosol sprayer with an even back and forth spray pattern to ensure uniformity, and the addon was controlled by weight using a top loading balance to ±3% accuracy.

The sodium carboxymethyl cellulose (CMC) was added during the manufacture of the airlaid dry basesheet. The basesheet was airlaid with a basis weight of about 60 g/m2 and the caliper was about 0.6 mm to allow for adequate penetration. Carboxymethyl cellulose (CMC) was supplied as a 4% Amtex Gelycel P2-10C solution dissolved in a 55 gallon tank. The sprayer was a single spray tip in a pilot line about 24″ wide. The pressure to deliver 2.00% CMC (as dried finished basesheet) was 40 psi; the pressure to deliver 2.45% CMC was 60 psi; and the pressure to deliver 2.9% CMC was 80 psi. The CMC can be made at a variety of concentrations and delivered at a variety of pressures, as long as they are sprayable and well distributed in the basesheet. The pulp used was Leaf River (LR 90) Grade 4725 semi-treated pulp that was hammermilled and delivered to 3 forming heads at a rate of (275-375 m/min).

A Response Surface Methodology (RSM) software was used with a central composite CCD design with statistical analysis software to analyze the variables. CCD designs can aid in optimizing compositions when the optimum point in the variable space is not known. A Box-Behnken design requires fewer samples, but can only optimize variables in the design of experiments (DOE) field. The responses tested were ball burst or poke-through force (Example 7); machine direction (MD) and cross direction (CD) tensile strength (Example 8) and water dispersibility (Example 9).

### Example 7: Strength Testing—Ball Burst Force (Samples 1-16)

This Example tested the strength of lotioned wipes (Samples 1-16) of Example 6 as measured by ball burst force as provided below.

Samples 1-16 were tested according to INDA Wiper Ball Burst Test Method WSP 110.5 R4 (12), which is incorporated by reference herein its entirety and provided in Example 3, to test the ball burst force. The ball burst force measurement is a measure of the force to poke a steel ball through the wet wipe. The higher the ball burst force measurement, the stronger the wipe material. The ball burst force is considered an accurate measurement since the sample size was 4″×6″ and the cut was far from the measurement point.

The CCD testing was analyzed using response surface methodology statistical analysis software to analyze ball burst force (lb.) versus carboxymethyl cellulose (CMC), butylene glycol and calcium chloride concentration of the lotioned wipes (Samples 1-16). The Ball Burst Response Surface has an R²=0.98 and a P Value=0.0002. P typically less than or equal to 0.05 are considered statistically significant.

To create appealing wipes to consumers, two main parameters can be considered:

Maximum Strength:

Use of humectant, salt, and binder to yield maximum ball burst force.

Maximum Mildness:

Use humectant, salt, and binder to yield maximum mildness.

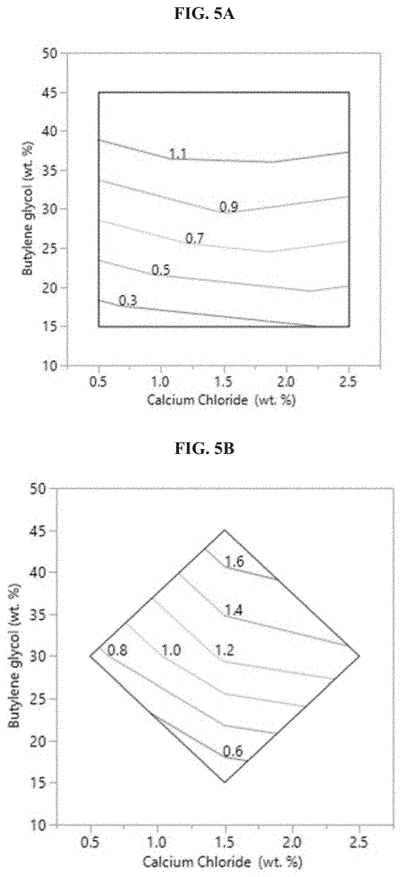

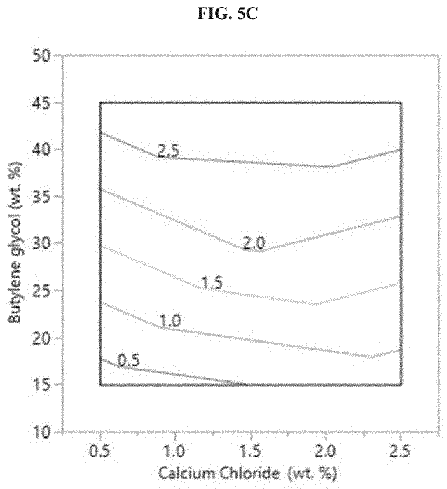

The results of the ball burst measurements are provided in Table 17. Contour plots based on the data of Table 17 (ball burst force (lb.)) are provided as FIGS. 5A-5C. FIG. 5A provides the calcium chloride and butylene glycol concentration versus ball burst force (lb.) at 2.00% carboxymethyl cellulose (CMC). FIG. 5B provides the calcium chloride and butylene glycol concentration versus ball burst force (lb.) at 2.45% carboxymethyl cellulose (CMC). FIG. 5C provides the calcium chloride and butylene glycol concentration versus ball burst force (lb.) at 2.90% carboxymethyl cellulose (CMC).

TABLE 17

| | Butylene | Calcium | | Ball Burst | Ball Burst |
|---|---|---|---|---|---|
| | Glycol | Chloride | CMC | Force | Dry Retention |
| Sample | (wt. %) | (wt. %) | (wt. %) | (lb.) | (% Dry) |
| 1 | 45 | 1.5 | 2.45 | 1.75 | 59 |
| 2 | 15 | 2.5 | 2.90 | 0.74 | 19 |
| 3 | 45 | 0.5 | 2.90 | 2.77 | 73 |
| 4 | 30 | 1.5 | 2.45 | 1.27 | 43 |
| 5 | 45 | 0.5 | 2.00 | 1.34 | 65 |
| 6 | 30 | 1.5 | 2.45 | 1.20 | 40 |
| 7 | 15 | 0.5 | 2.90 | 0.27 | 7 |
| 8 | 15 | 1.5 | 2.45 | 0.44 | 15 |
| 9 | 30 | 1.5 | 2.90 | 2.08 | 55 |
| 10 | 45 | 2.5 | 2.90 | 2.86 | 75 |
| 11 | 30 | 0.5 | 2.45 | 0.73 | 25 |
| 12 | 45 | 2.5 | 2.00 | 1.37 | 66 |
| 13 | 15 | 2.5 | 2.00 | 0.32 | 15 |
| 14 | 15 | 0.5 | 2.00 | 0.17 | 8 |
| 15 | 30 | 2.5 | 2.45 | 1.37 | 46 |
| 16 | 30 | 1.5 | 2.00 | 0.92 | 44 |

The dielectric constant of the lotion reduced the solubility of the carboxymethyl cellulose (CMC). The CCD testing showed that the concentration of butylene glycol in the lotion is highly significant (P=0.0001); the carboxymethyl cellulose (CMC) concentration in the basesheet is highly significant (P=0.00019); the interaction between butylene glycol and carboxymethyl cellulose (CMC) is highly significant (P=0.00328); and the level of calcium chloride is also significant (P=0.05162).

As provided in FIGS. 5A-5C, as the level of carboxymethyl cellulose (CMC) increases, the sheet strength of the sample increases. Butylene glycol and carboxymethyl cellulose (CMC) highly increased the sheet strength. Since carboxymethyl cellulose (CMC) is a binder, the more binder added the stronger the sheet so long as the binder adequately penetrated the sheet. Since butylene glycol reduces the solubility of the binder, the higher the butylene glycol concentration the stronger the sheet.

Calcium chloride or sodium can be used to decrease the solubility of sodium carboxymethyl cellulose (CMC). The absence of salt can cause the ball burst for force to be immeasurably small. For this reason, the lower limit of calcium chloride was set to 0.5% in the design of experiments (DOE). Calcium can replace the sodium and more efficiently increase strength than sodium. The importance of calcium increases as the level of dielectric constant modifying solvent decreases as shown in Table 18.

The effect of calcium chloride on ball burst force for at high (i.e., 45%) and low (i.e., 15%) butylene glycol concentrations is provided in Table 18.

TABLE 18

| Carboxymethyl | Butylene | Calcium | |
|---|---|---|---|
| Cellulose (CMC) | Glycol | Chloride | Ball Burst |
| Concentration | Concentration | Concentration | Force |
| (%) | (%) | (%) | (lb.) |
| 2.9 | 15 | 0 | <0.1 |
| 2.9 | 15 | 0.50 | 0.33 |
| 2.9 | 15 | 1.50 | 0.74 |
| 2.9 | 15 | 2.50 | 0.82 |
| 2.9 | 45 | 0.50 | 2.68 |
| 2.9 | 45 | 1.50 | 2.97 |
| 2.9 | 45 | 2.50 | 2.99 |

Dry strength retention of dry strength is also a useful measure of premoistened wipes. Typically, when dispersible wipes sheets are moistened, much of the dry strength is lost. Surprisingly, the dry strength retention was relatively high for the highly dispersible wipes of the present disclosure.

The dry sheet strength in terms of ball burst force versus carboxymethyl cellulose (CMC) concentration is provided in Table 19.

TABLE 19

| Carboxymethyl Cellulose | Ball Burst |
|---|---|
| (CMC) Concentration | Force |
| (% of dry sheet weight) | (lb.) |
| 2.00 | 2.07 |
| 2.45 | 2.97 |
| 2.90 | 3.80 |

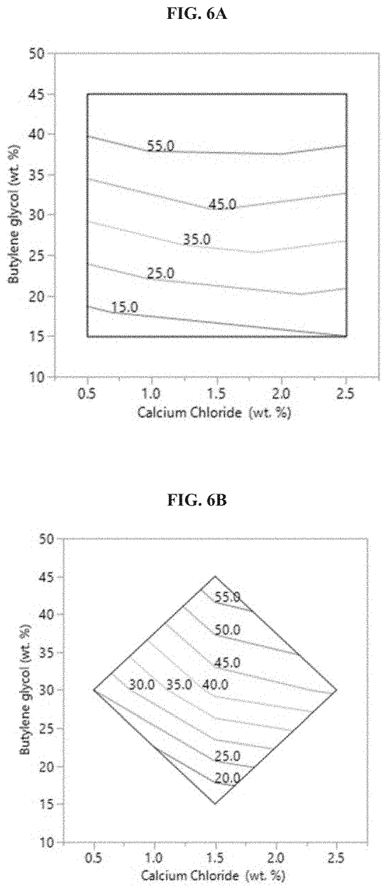

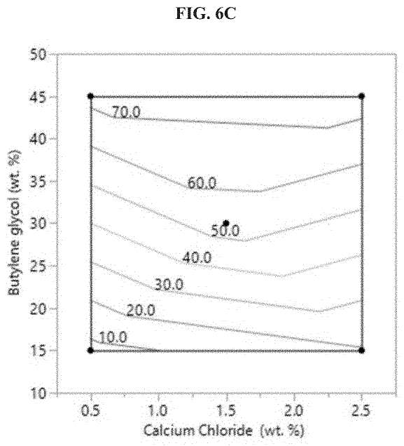

Contour plots based on the data of Table 17 (ball burst force—dry retention (% dry)) are provided as FIGS. 6A-6C. FIG. 6A provides the calcium chloride and butylene glycol concentration versus ball burst force—dry retention (% dry) at 2.00% carboxymethyl cellulose (CMC). FIG. 6B provides the calcium chloride and butylene glycol concentration versus ball burst force—dry retention (% dry) at 2.45% carboxymethyl cellulose (CMC). FIG. 6C provides the calcium chloride and butylene glycol concentration versus ball burst force—dry retention (% dry) at 2.90% carboxymethyl cellulose (CMC). As the butylene glycol concentration increases the wet retention increases, which can be attributable in part to two factors. First, the solubility of the binder, carboxymethyl cellulose (CMC), decreases. Second, the hydrogen bonding of the fibers is protected, which can also strength. Testing on the basesheet with no butylene glycol was not performed because the sheet disintegrated and had no measurable ball burst force.

### Example 8: Strength Testing—Cross-Machine Direction (CD) and Machine Direction (MD) Tensile Strength (Samples 1-16)

This Example tested the strength of lotioned wipes (Samples 1-16) of Example 6 as measured by cross-machine direction (CD) tensile strength and machine direction (MD) tensile strength as provided below.

Tensile or the force per linear inch or grams per linear inch (gli) was measured for airlaid wipes in the machine direction (MD) and cross-direction (CD). The direction the fabric was moving was the MD and the direction across the fabric, perpendicular to the MD, was the CD.

The tensile measurements were made with an Instron Tensile Tester using Instron Series IX software following new TAPPI test method T 576 pm-07. Handling during cutting or cutting with dull scissors can damage the sheet and reduce tensile measurements.

The CCD experiment was analyzed using JMR Version 13 DOE RSM statistical analysis software to analyze CD and MD tensile strength concurrently versus carboxymethyl cellulose (CMC), butylene glycol and calcium chloride concentration. The MD and CD tensile, when optimized together, has a Response Surface with an R²=0.99 and a P Value=0.0001. P typically less than or equal to 0.05 are considered statistically significant.

The dielectric constant of the lotion was reduced to reduce the solubility of the carboxymethyl cellulose (CMC) and increase the strength. The CCD experiment showed that the concentration of butylene glycol in the lotion is highly significant (P=0.0000); the CMC concentration in the basesheet is highly significant (P=0.00004); the interaction between butylene glycol and CMC is highly significant (P=0.00056); and the level of calcium chloride is highly significant (P=0.01090).

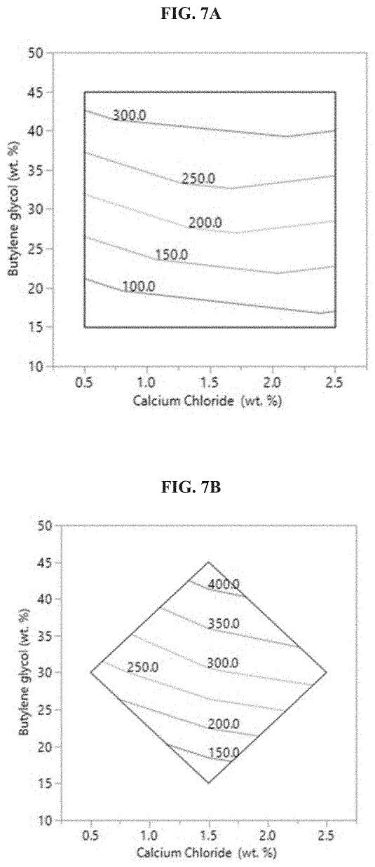

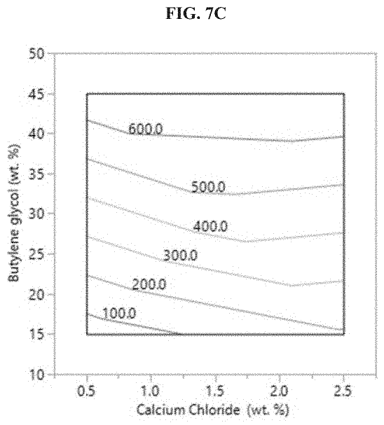

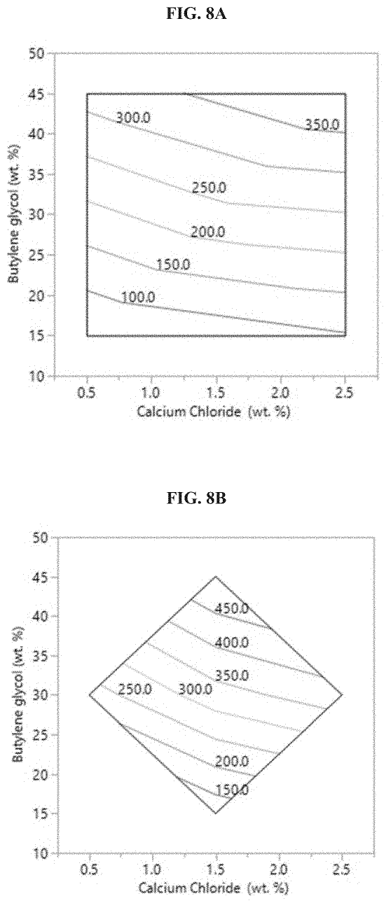

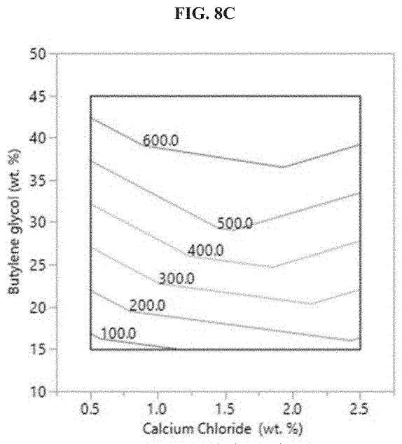

The results of the CD and MD tensile strength measurements are provided in Table 20. Contour plots based on the data of Table 20 (CD tensile strength (gli)) are provided as FIGS. 7A-7C. FIG. 7A provides the calcium chloride and butylene glycol concentration versus CD tensile strength (gli) at 2.00% carboxymethyl cellulose (CMC). FIG. 7B provides the calcium chloride and butylene glycol concentration versus CD tensile strength (gli) at 2.45% carboxymethyl cellulose (CMC). FIG. 7C provides the calcium chloride and butylene glycol concentration versus CD tensile strength (gli) at 2.90% carboxymethyl cellulose (CMC). Contour plots based on the data of Table 20 (MD tensile strength (gli)) are provided as FIGS. 8A-8C. FIG. 8A provides the calcium chloride and butylene glycol concentration versus MD tensile strength (gli) at 2.00% carboxymethyl cellulose (CMC). FIG. 7B provides the calcium chloride and butylene glycol concentration versus MD tensile strength (gli) at 2.45% carboxymethyl cellulose (CMC). FIG. 7C provides the calcium chloride and butylene glycol concentration versus MD tensile strength (gli) at 2.90% carboxymethyl cellulose (CMC).

TABLE 20

| | Butylene | Calcium | CMC | CD | CD | MD | MD |
|---|---|---|---|---|---|---|---|
| Sam- | Glycol | Chloride | (wt. | Tensile | Tensile | Tensile | Tensile |
| ple | (wt. %) | (wt. %) | %) | (gli) | (% Dry) | (gli) | (% Dry) |
| 1 | 45 | 1.5 | 2.45 | 434.4 | 36 | 504.4 | 34 |
| 2 | 15 | 2.5 | 2.90 | 189.8 | 10 | 177.0 | 9 |
| 3 | 45 | 0.5 | 2.90 | 669.0 | 36 | 651.2 | 34 |
| 4 | 30 | 1.5 | 2.45 | 306.9 | 25 | 322.6 | 22 |
| 5 | 45 | 0.5 | 2.00 | 322.1 | 39 | 320.5 | 35 |
| 6 | 30 | 1.5 | 2.45 | 282.2 | 23 | 335.97 | 23 |
| 7 | 15 | 0.5 | 2.90 | 47.7 | 3 | 63.5 | 3 |
| 8 | 15 | 1.5 | 2.45 | 106.8 | 9 | 116.1 | 8 |
| 9 | 30 | 1.5 | 2.90 | 463.9 | 25 | 522.2 | 27 |
| 10 | 45 | 2.5 | 2.90 | 690.3 | 37 | 702.4 | 36 |
| 11 | 30 | 0.5 | 2.45 | 229.4 | 19 | 225.9 | 15 |
| 12 | 45 | 2.5 | 2.00 | 343.6 | 41 | 399.1 | 43 |
| 13 | 15 | 2.5 | 2.00 | 82.9 | 10 | 96.3 | 10 |
| 14 | 15 | 0.5 | 2.00 | 42.5 | 5 | 49.5 | 5 |
| 15 | 30 | 2.5 | 2.45 | 325.2 | 27 | 381.5 | 26 |
| 16 | 30 | 1.5 | 2.00 | 230.1 | 28 | 234.9 | 25 |

FIGS. 7A-7C and 8A-8C illustrate that as the carboxymethyl cellulose (CMC) concentration increases, the sheet strength increases. Butylene glycol and carboxymethyl cellulose (CMC) both provide the calcium chloride and butylene glycol concentration versus CD tensile (gli) at 2.00% carboxymethyl cellulose (CMC). FIG. 7B provides the calcium chloride and butylene glycol concentration versus CD tensile (gli) at 2.45% carboxymethyl cellulose (CMC). FIG. 7C provides the calcium chloride and butylene glycol concentration versus CD tensile (gli) at 2.90% carboxymethyl cellulose (CMC) increase the strength of the sheet. Since carboxymethyl cellulose (CMC) is a binder, the more binder was added, the stronger sheet. Since butylene glycol reduces the solubility of the binder and increases the hydrogen bonding of the fibers, the higher the butylene glycol concentration, the stronger the sheet. The carboxymethyl cellulose (CMC) to butylene glycol correlation can be the strongest in increasing strength of the sheet. The calcium addition improved the strength further without impacting dispersibility. The combination of calcium chloride and butylene glycol allowed for lower butylene glycol concentrations and a reduced cost sheet.

Wet retention of dry strength was further measured. Typically, when dispersible wipes sheets are moistened the dry strength is lost. Surprisingly, the wet strength retention highly increased in the same manner as the ball burst force. The dry sheet strength results in terms of tensile versus carboxymethyl cellulose (CMC) concentration are provided in Table 21.

TABLE 21

| Carboxymethyl Cellulose | CD Tensile | MD Tensile |
|---|---|---|
| (CMC) Concentration | Strength | Strength |
| (% of dry sheet weight) | (gli) | (gli) |
| 2.00 | 836 | 922 |
| 2.45 | 1,209 | 1,512 |
| 2.90 | 1,842 | 1,927 |

Dividing the tensile strength from the data and multiplying by 100% yields similar results to only the maximum dry strength retention was slightly above 40%. This reduction is believed to be caused by the low stretch of carboxymethyl cellulose (CMC) basesheets combined with the variability caused by cutting the 1.5″×1.0″ strips used for tensile testing vs. the 6″×4″ pieces used for the ball burst testing.

### Example 9: Dispersibility Testing (Samples 1-16)

This Examples tested lotions on wipe materials of Example 6 (Samples 1-16) for dispersibility in comparison to commercial products. Commercial products included Cottonelle® Flushable Wipes; Charmin® Flushable Wipes; Great Value® Flushable Wipes; and Kroger® Home Sense™ Flushable Wipes.

Water dispersibility testing was performed with room temperature water and a slosh box apparatus (Research Dimensions, Neenah, Wis.); a 12.5 mm screen; and a Peerless showerhead (Model 76114WH). The pre-moistened wipes were dried in an oven at 105° C. overnight and the basesheet was weighed. Six (6) replicates of each pre-moistened wipe were tested in the sloshbox for 1 minute at 26 rpm and in 2 L of water. The sample was filtered with 12.5 mm screen and the screen was rinsed with 4 L/min water with the spray at 6 in. above the screen for 2 minutes. The residual wipe was removed from the screen and dried overnight at 105° C. The dried residual wipe was weighed. To calculate a residual percentage, the dried residual wipe weight was divided from the dried wipe weight and multiplied by 100%. The dispersibility percentage was 100% of the residual percentage. The 1-minute test is predictive of wipes that can break apart prior to reaching a sewage system.

Results of the dispersibility testing are provided in Table 22. Samples 1-16 completely dispersed within 1 minute.

TABLE 22

| Sample | Dispersibility (%) |
|---|---|
| Samples 1-16 | 100.0 |
| Cottonelle ® Flushable Wipes | 0.6 |
| Charmin ® Flushable Wipes | 5.5 |
| Great Value ® Flushable Wipes | 6.0 |
| Kroger ® Home Sense ™ Flushable Wipes | 6.7 |

### Example 10: Maximum Mildness and Reduced Costs

This Example evaluated the maximum mildness and lowest cost for carboxymethyl cellulose (CMC) wipes.

Maximum mildness and lowest cost for carboxymethyl cellulose (CMC) wipes was observed when the butylene glycol and calcium chloride are minimized. Ball burst force (lb.); machine direction (MD) tensile strength (gli); and cross-machine direction (CD) tensile strength (gli) can be minimized. A functional strength minimum for handling of carboxymethyl cellulose (CMC) wipes is as follows:

Ball Burst Force=0.41 lb.

CD Tensile Strength=100 gli

MD Tensile Strength=100 gli

At values below these strengths, it can be difficult to convert, dispense, and use carboxymethyl cellulose (CMC) wipes. Using a prediction profiler in RS1 to solve for the minimum, the model at the calcium chloride inflection point yielded the results provided in Table 23.

TABLE 23

| Carboxymethyl | Butylene Glycol | Calcium Chloride |
|---|---|---|
| cellulose (CMC) | Concentration | Concentration |
| Level (wt. %) | (wt. %) | (wt. %) |
| 2.00 | 15.5 | 1.6 |
| 2.45 | 16.8 | 1.5 |
| 2.90 | ~13.0 | 1.4 |

As provided in Table 23, at 2.9% carboxymethyl cellulose (CMC), the concentration of carboxymethyl cellulose (CMC) rises to the point that it has reduced solubility in the butylene glycol composition, thereby imparting outsized strength gains in the presence of low carboxymethyl cellulose (CMC) concentration.

Optimizing mildness and strength wipes can occur when the butylene glycol and calcium chloride are balanced for consumer use. Ball burst force (lb.); machine direction (MD) tensile strength (gli); and cross-machine direction (CD) tensile strength (gli) can be minimized. A functional strength minimum for handling carboxymethyl cellulose (CMC) wipes is as follows:

Ball Burst Force=1.20 lb.

CD Tensile Strength=250 gli

MD Tensile Strength=250 gli

At values below these strengths, it can be difficult to convert, dispense, and use carboxymethyl cellulose (CMC) wipes. Using a prediction profiler in RS1 to solve for the minimum, the model at the calcium chloride inflection point yielded the results provided in Table 24.

TABLE 24

| Carboxymethyl | Butylene Glycol | Calcium Chloride |
|---|---|---|
| Cellulose (CMC) | Concentration | Concentration |
| Level (wt. %) | (wt. %) | (wt. %) |
| 2.00 | 35.0 | 1.45 |
| 2.45 | 29.6 | 1.50 |
| 2.90 | 20.3 | 1.55 |

As provided in Table 24, at 2.9% carboxymethyl cellulose (CMC), the concentration of carboxymethyl cellulose (CMC) rises to the point that it has reduced solubility in the butylene glycol composition, thereby imparting outsized strength gains in the presence of low carboxymethyl cellulose (CMC) concentration.

### Example 11: Dielectric Constant of Lotion Formulations and Solubility of Water Soluble Binders (Lotions 66-81)

This Example evaluated how decreasing the dielectric constant of lotion formulations synergistically decreased the solubility of water soluble binders, such as carboxymethyl cellulose (CMC), thereby strengthening the sheet.

All lotions (Lotions 66-81) included the base formulation provided in Table 25.

TABLE 25

| Component | | Solids (wt-%) |
|---|---|---|
| Deionized Water | Solvent | 45.34 |
| CaCl₂ * 2H₂O | Sheet | 0.00 |
| | Strengthening | |
| | Agent | |
| Butylene Glycol | Solvent DEC | 0.00 |
| | Reducer | |
| Hexylene Glycol | Solvent DEC | 5.00 |
| | Reducer | |
| Polysorbate 20 | Coupling Agent | 0.50 |
| Phenoxy Ethanol | Preservative | 0.80 |
| Fragrance | | 0.05 |
| Total | | 100 |
| pH | | 5.20 |

The base formulation for Lotions 66-81 was adjusted to include calcium chloride (CaCl₂)) in an amount of 0%, 0.5% 1.5%, or 2.5% and butylene glycol in an amount of 0%, 15%, 30% or 45% as provided in Table 26.

TABLE 26

| | Butylene Glycol | Calcium Chloride |
|---|---|---|
| Lotion | (wt.-%) | (wt.-%) |
| 66 | 0% | 0% |
| 67 | 0% | 0.5% |
| 68 | 0% | 1.5% |
| 69 | 0% | 2.5% |
| 70 | 15% | 0% |
| 71 | 15% | 0.5% |
| 72 | 15% | 1.5% |
| 73 | 15% | 2.5% |
| 74 | 30% | 0% |
| 75 | 30% | 0.5% |
| 76 | 30% | 1.5% |
| 77 | 30% | 2.5% |
| 78 | 45% | 0% |
| 79 | 45% | 0.5% |
| 80 | 45% | 1.5% |
| 81 | 45% | 2.5% |

For each lotion formulation, 1 g to 1.9 g Gelycel® PC-10 sodium carboxymethyl cellulose (CMC) was added to 200 g lotion and mixed vigorously with 1″ stir bar for 5 min. The lotion was rested for 1 hour. The lotion was filtered with 1.5 μm (Whatman 934-AH) filter, while rinsing with butylene glycol. The lotion was dry filtered overnight at 105° C. The residue weight for each lotion sample was determined. The filtrate recovery was calculated as a percentage of the sodium carboxymethyl cellulose (CMC) that was initially added to the lotion. Each lotion was tested twice.

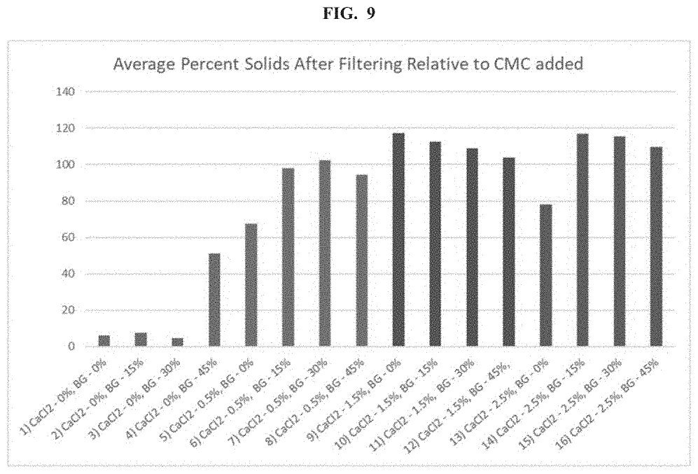

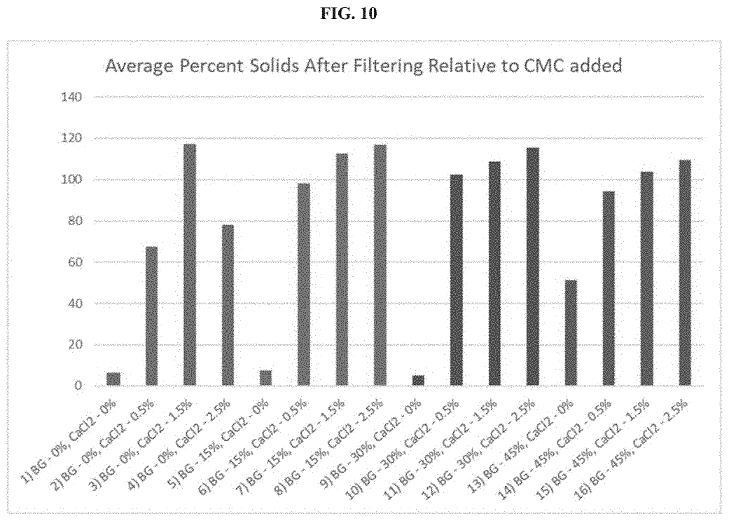

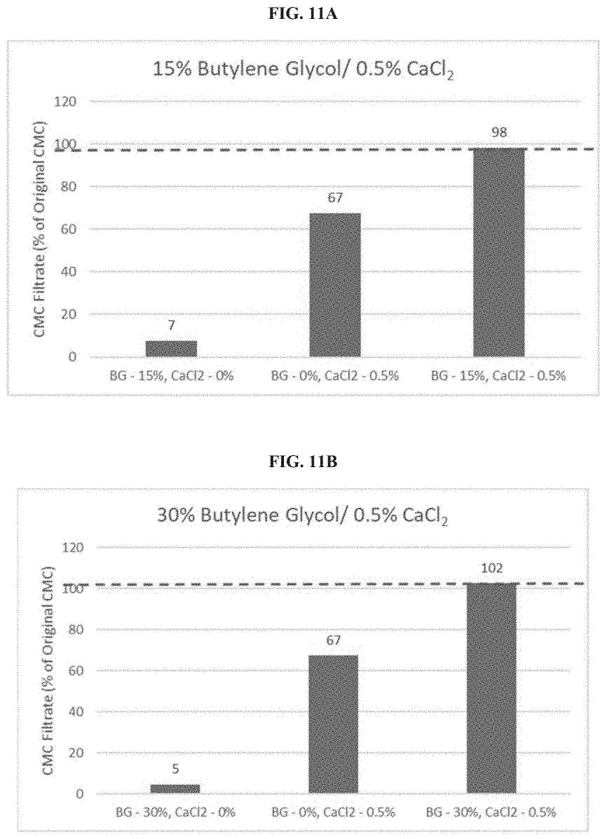

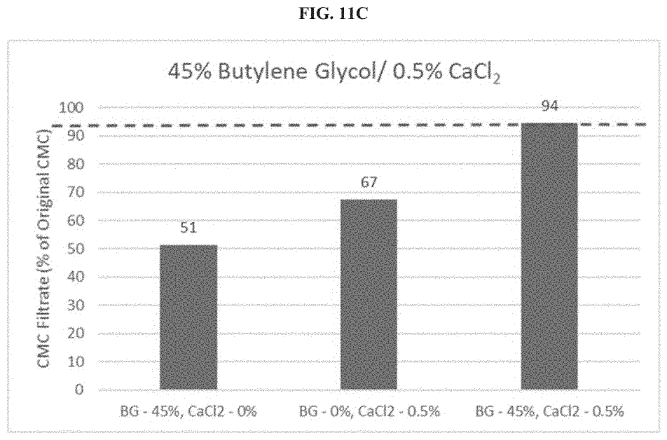

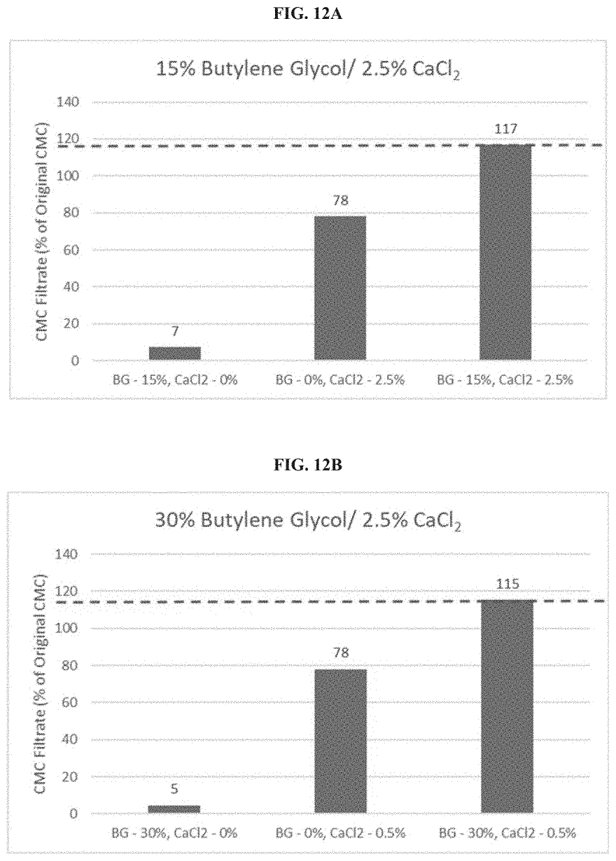

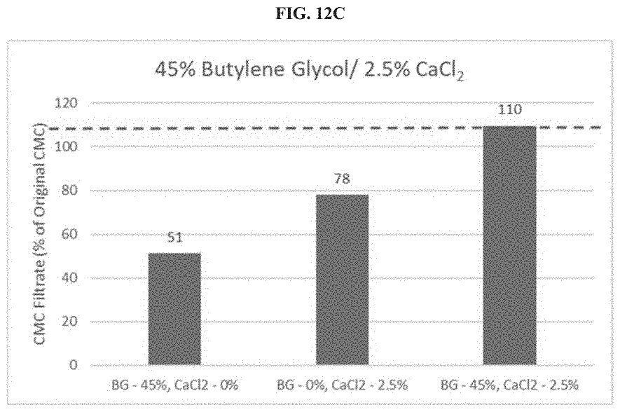

The results are provided in Tables 27 and 28 and FIGS. 9, 10, 11A-11C, and 12A-12C. FIG. 9 provides the average percent solids after filtering relative to carboxymethyl cellulose (CMC) added in order of increasing calcium chloride concentration. FIG. 10 provides the average percent solids after filtering relative to carboxymethyl cellulose (CMC) added in order of increasing butylene glycol concentration. FIGS. 11A-11C provide the carboxymethyl cellulose (CMC) filtrate (% of original CMC) for 0.5% calcium chloride and 15% butylene glycol; 30% butylene glycol; and 45% butylene glycol, respectively. FIGS. 12A-12C provide the carboxymethyl cellulose (CMC) filtrate (% of original CMC) for 2.5% calcium chloride and 15% butylene glycol; 30% butylene glycol; and 45% butylene glycol, respectively.

TABLE 27

| | | | | | | | Percent | | |
|---|---|---|---|---|---|---|---|---|---|
| | | | | Tin/ | | | Remaining | | |
| | | | Total | Filter | | | Relative to | Percent | Initial |
| | Weight | | Sample | Tare | Dried | | CMC | CaCl2 in | Weight of |
| | CMC | Weight of | Weight | Weight | Weight | Remaining | amount | Lotion | CaCl₂ in |
| Lotion | (g) | Lotion (g) | (g) | (g) | (g) | Solids (g) | (%) | (%) | Lotion (g) |
| 66 | 1.4495 | 100.0172 | 101.4667 | 2.9129 | 3.0006 | 0.0877 | 6.05 | 0 | 0 |
| 66 | 1.0076 | 100.0255 | 101.0331 | 2.8953 | 2.9537 | 0.0584 | 5.79 | 0 | 0 |
| 67 | 0.7247 | 50.0189 | 50.7436 | 2.4147 | 2.9441 | 0.5294 | 73.05 | 0.5 | 0.2500945 |
| 67 | 0.5092 | 50.0506 | 50.5598 | 2.424 | 2.738 | 0.314 | 61.66 | 0.5 | 0.250253 |
| 68 | 1.0022 | 100.0528 | 101.055 | 2.9492 | 4.124 | 1.1748 | 117.22 | 1.5 | 1.500792 |
| 68 | 1.4542 | 100.0087 | 101.4629 | 2.875 | 3.4147 | 0.5397 | 37.11 | 1.5 | 1.5001305 |
| 69 | 1.4519 | 100.2379 | 101.6898 | 2.9077 | 4.0979 | 1.1902 | 81.97 | 2.5 | 2.5059475 |
| 69 | 1.0055 | 100.2905 | 101.296 | 2.9142 | 3.6587 | 0.7445 | 74.042 | 2.5 | 2.5072625 |
| 70 | 1.4604 | 100.349 | 101.8094 | 2.8693 | 2.9536 | 0.0843 | 5.77 | 0 | 0 |
| 70 | 0.9979 | 100.3247 | 101.3226 | 2.8926 | 2.9834 | 0.0908 | 9.09 | 0 | 0 |
| 71 | 1.446 | 100.4342 | 101.8802 | 2.8832 | 4.2406 | 1.3574 | 93.87 | 0.5 | 0.502171 |
| 71 | 0.9984 | 100.0348 | 101.0332 | 2.915 | 3.9377 | 1.0227 | 102.43 | 0.5 | 0.500174 |
| 72 | 1.4593 | 100.02 | 101.4793 | 2.8925 | 4.4763 | 1.5838 | 108.53 | 1.5 | 1.5003 |
| 72 | 1.0087 | 100.1875 | 101.1962 | 2.9182 | 4.0953 | 1.1771 | 116.69 | 1.5 | 1.5028125 |
| 73 | 1.4553 | 100.7823 | 102.2376 | 2.9309 | 4.59 | 1.6591 | 114.00 | 2.5 | 2.5195575 |
| 73 | 1.0083 | 100.3786 | 101.3869 | 2.8671 | 4.0721 | 1.205 | 119.50 | 2.5 | 2.509465 |
| 74 | — | — | — | — | — | — | — | — | 0 |
| 74 | 1.0011 | 100.0176 | 101.0187 | 2.3997 | 2.4468 | 0.0471 | 4.70 | 0 | 0 |
| 75 | 1.4508 | 100.0413 | 101.4921 | 2.9145 | 4.3942 | 1.4797 | 101.99 | 0.5 | 0.5002065 |
| 75 | 1.0036 | 100.2583 | 101.2619 | 2.9075 | 3.9401 | 1.0326 | 102.88 | 0.5 | 0.5012915 |
| 76 | 1.4485 | 99.9365 | 101.385 | 2.8514 | 4.3843 | 1.5329 | 105.82 | 1.5 | 1.4990475 |
| 76 | 1.0067 | 100.2663 | 101.273 | 2.8927 | 4.0181 | 1.1254 | 111.79 | 1.5 | 1.5039945 |
| 77 | 1.4489 | 100.0832 | 101.5321 | 2.8903 | 4.4933 | 1.603 | 110.63 | 2.5 | 2.50208 |
| 77 | 1.0029 | 100.379 | 101.3819 | 2.8697 | 4.0735 | 1.2038 | 120.031 | 2.5 | 2.509475 |
| 78 | 1.4524 | 100.0134 | 101.4658 | 2.3912 | 3.1728 | 0.7816 | 53.81 | 0 | 0 |
| 78 | 1.0029 | 100.0939 | 101.0968 | 2.4398 | 2.9289 | 0.4891 | 48.76 | 0 | 0 |
| 79 | 1.4582 | 100.7037 | 102.1619 | 2.9101 | 4.4078 | 1.4977 | 102.70 | 0.5 | 0.5035185 |
| 79 | 1.004 | 100.2049 | 101.2089 | 2.8574 | 3.7216 | 0.8642 | 86.07 | 0.5 | 0.5010245 |
| 80 | 1.4509 | 100.6 | 102.0509 | 2.883 | 4.355 | 1.472 | 101.45 | 1.5 | 1.509 |
| 80 | 1.0084 | 100.2108 | 101.2192 | 2.8921 | 3.9615 | 1.0694 | 106.049 | 1.5 | 1.503162 |
| 81 | 1.4568 | 100.0916 | 101.5484 | 2.8517 | 4.4003 | 1.5486 | 106.30 | 2.5 | 2.50229 |
| 81 | 1.0057 | 100.8708 | 101.8765 | 2.9155 | 4.0504 | 1.1349 | 112.84 | 2.5 | 2.52177 |

TABLE 28

| | | | Average Percent Solids After |
|---|---|---|---|
| | Butylene | Calcium | Filtering Relative to |
| | Glycol | Chloride | Carboxymethyl cellulose |
| Lotion | (wt.-%) | (wt.-%) | (CMC) Added (%) |
| 66 | 0% | 0% | 5.92 |
| 67 | 0% | 0.5% | 67.35 |
| 68 | 0% | 1.5% | 117.22 |
| 69 | 0% | 2.5% | 78.00 |
| 70 | 15% | 0% | 7.43 |
| 71 | 15% | 0.5% | 98.15 |
| 72 | 15% | 1.5% | 112.61 |
| 73 | 15% | 2.5% | 116.75 |
| 74 | 30% | 0% | 4.70 |
| 75 | 30% | 0.5% | 102.44 |
| 76 | 30% | 1.5% | 108.80 |
| 77 | 30% | 2.5% | 115.33 |
| 78 | 45% | 0% | 51.29 |
| 79 | 45% | 0.5% | 94.39 |
| 80 | 45% | 1.5% | 103.75 |
| 81 | 45% | 2.5% | 109.57 |

As shown in FIG. 10, lotions having at least 5% hexylene glycol, 15% butylene glycol and 1.5% calcium chloride provided sufficient insolubility of the carboxymethyl cellulose (CMC) binder. Butylene glycol concentrations of greater than 15% (i.e., at 30% and 45%) further provided some additional increase in insolubility of the carboxymethyl cellulose (CMC) binder.

Lotion 68, represented as number 3 in FIG. 10, included 0% butylene glycol and 1.5% calcium chloride. During testing of Lotion 68, an error was identified in the filtration of the lotion based on the amount of percent solids obtained. Lotion 68 is to be prepared and retested (as Lotion 68′). Lotion 68′ is expected to follow the same trends as shown in FIG. 10 as the additional butylene glycol concentrations. For example, Lotion 68′ is expected to have a value of average percent solids after filtering relative to carboxymethyl cellulose (CMC) added (%) between the measured values for Lotions 67 and 69, i.e., between 67.35% and 78.00%. Further testing on samples with respect to filtration is to be conducted. Additionally, further testing on samples with respect to butylene glycol concentrations between 0% and 15% is to be conducted as noted in Example 12.

Lotions with calcium chloride (CaCl₂)) caused calcium to replace the sodium in the carboxymethyl cellulose (CMC). Since the atomic mass of calcium is 40.1 and the atomic mass of sodium is 23.0, the molecular weight of calcium carboxymethyl cellulose (CMC) is higher than the molecular of sodium carboxymethyl cellulose (CMC). As shown in Table 28 and FIGS. 11A-11C and 12A-12C, the recovery % of calcium carboxymethyl cellulose (CMC) can exceed 100% of the initial sodium carboxy methyl cellulose (CMC) due to the differences in molecular weight. It is also possible that calcium carboxymethyl cellulose (CMC) contains associated water. As provided in Table 28 and FIGS. 11A-11C and 12A-12C, it is demonstrated that calcium is replacing the sodium in carboxymethyl cellulose (CMC) and that the combination of butylene glycol and calcium chloride (CaCl₂)) synergistically decreases the solubility of carboxymethyl cellulose (CMC). The error for this type of measurement can be typically ±6% when the recovery is 50% or greater, and can be as high as ±3% when the recovery is under 10%.

Decreasing the dielectric constant of lotion formulations was determined to synergistically decrease the solubility of carboxymethyl cellulose (CMC), a water-soluble binder. With decreased solubility of the binder, the sheet is therefore strengthened. With increasing polarity, hydrogen bonding is destroyed resulting in a weaker sheet. Salts, such as calcium carbonate, can increase sheet strength whereas the dielectric constant (DEC) can lower sheet strength. Accordingly, it was surprisingly determined that adding salts, such as calcium chloride (CaCl2), which would expectedly increase the dielectric constant (DEC) of a solution and weaken sheet strength, in contrast, provided for increased insolubility and increased sheet strength.

### Example 12: Dielectric Constant of Lotion Formulations and Solubility of Water Soluble Binders (Lotions 82-93)

This Example evaluates how decreasing the dielectric constant of lotion formulations synergistically decreased the solubility of water soluble binders, such as carboxymethyl cellulose (CMC), thereby strengthening the sheet. Such lotion formulations include butylene glycol at a concentration between 0% and 15%.

All formulations (Lotions 82-93) include the base formulation of Table 25 in Example 11. The base formulation for Lotions 82-93 is adjusted to include calcium chloride (CaCl₂)) in an amount of 0%, 0.5%, 1.5%, or 2.5% and butylene glycol in an amount of 1%, 5%, or 10% as provided in Table 29.

TABLE 29

| | Butylene Glycol | Calcium Chloride |
|---|---|---|
| Lotion | (wt.-%) | (wt.-%) |
| 82 | 1% | 0% |
| 83 | 1% | 0.5% |
| 84 | 1% | 1.5% |
| 85 | 1% | 2.5% |
| 86 | 5% | 0% |
| 87 | 5% | 0.5% |
| 88 | 5% | 1.5% |
| 89 | 5% | 2.5% |
| 90 | 10% | 0% |
| 91 | 10% | 0.5% |
| 92 | 10% | 1.5% |
| 93 | 10% | 2.5% |

For each lotion formulation, 1 g to 1.9 g Gelycel® PC-10 sodium carboxymethyl cellulose (CMC) is added to 200 g lotion and mixed vigorously with 1″ stir bar for 5 min. The lotion is rested for 1 hour. The lotion is filtered with 1.5 μm (Whatman 934-AH) filter, while rinsing with butylene glycol. The lotion is dry filtered overnight at 105° C. The residue weight for each lotion sample is determined. The filtrate recovery is calculated as a percentage of the sodium carboxymethyl cellulose (CMC) that was initially added to the lotion. Each lotion is tested twice and measurements are taken as provided in Example 11.

A similar trend for Lotions 82-93 is expected as provided in Example 11, Table 28 and FIG. 10. Specifically, lotions with a concentration of butylene glycol between 0% and 15% (i.e., at 1%, 5%, and 10%) and a concentration of calcium chloride (i.e., at 0.5%, 1.5%, or 2.5%) will provide for increased insolubility of the binder, i.e., carboxymethyl cellulose (CMC) as compared to lotions with a calcium chloride (CaCl₂) concentration of 0%. Further, for each percentage of butylene glycol, it is expected that as the calcium chloride concentration increases (i.e., from 0.5% to 1.5% to 2.5%), the insolubility of the carboxymethyl cellulose (CMC) binder will increase. Further, it is expected that a further inflection point for increased insolubility of the carboxymethyl cellulose (CMC) binder, for lotion formulations with a butylene glycol concentration between 0% and 15% can be determined.

### Example 13: Personal Care Components (Skin Protectants) Evaluation

This Example evaluates personal care additives to lotions for use with dispersible wipes and in the solvent systems of the present disclosure. Such lotion additives can provide dispersible wipes with a dry powdery after-feel. Further, these additives have been surprisingly found to highly improve skin feel. The particular additives evaluated; potential function/mode of action; ranges evaluated; processing/stabilization; and observations are provided in Table 30.

TABLE 30

| | Function/Mode of | Ranges | | |
|---|---|---|---|---|
| Additive | Action | Evaluated | Processing/Stabilization | Observations |
| Lauroyl lysine | Small, waxy plates | 0.5-2% | Cold blend with | Provides good hand |
| | deposit on skin and | | remaining ingredients. | feel. |
| | slide past each | | Flakes settle out over | |
| | other giving a | | time. Would require a | |
| | smooth feeling. | | more viscous base to | |
| | | | suspend particles. | |
| Ethylene glycol | Commonly used as | 1-5% | Heat EGDS with | Provides some |
| disterate (EGDS) | a pearlescent, when | | surfactants to melt (~65° | improvement to hand |
| | properly prepared | | C.). Heat water to same | feel of base lotion. |
| | forms small, waxy | | temperature and add | |
| | flakes as above. | | EGDS/surfactant mix | |
| | | | with agitation. Allow to | |
| | | | slowly cool to below 40° | |
| | | | C. Add remaining | |
| | | | ingredients and mix to | |
| | | | uniformity. Properly | |
| | | | prepared suspensions | |
| | | | will keep EGDS | |
| | | | suspended for months at | |
| | | | room temperature. | |
| Polydimethylsiloxane | Oil-like liquid | 1-5% | Emulsification of PDMS | Provides good hand |
| (PDMS, silicone | coats skin and | | in the solvent system can | feel. |
| fluid, various | provides slip. | | be difficult. Mixtures of | Need to keep an |
| viscosities) | Lower MW, lower | | Tween ® and Span ® | emulsion or dispersion |
| | viscosity fluids | | emulsifiers gave short- | with the base lotion |
| | more volatile and | | term stability, but all of | stable as it starts to |
| | less substantive | | them separated within | offset improvements |
| | than higher MW, | | several hours after | in hand feel. |
| | higher viscosity | | making. Blend PDMS | |
| | fluids. Used in a | | with emulsifiers and | |
| | variety of personal | | other oily materials. | |
| | care products to | | Slowly add to aqueous | |
| | coat skin. | | phase with good | |
| | | | agitation. Increasing the | |
| | | | amount of emulsifier will | |
| | | | tend to increase the | |
| | | | stability of the emulsion, | |
| | | | but at the risk of the | |
| | | | system feeling “tacky” | |
| | | | during dry-down. | |
| Coconut oil, avocado | Natural oil coats | 1% | Warm coconut oil to | Good hand feel,. |
| oil, or olive oil (as | skin and provides | | melt (avocado and olive | Need to keep an |
| representatives of | slip. | | oil do not need to be | emulsion or dispersion |
| vegetable oils) | | | heated). Add | with the base lotion |
| | | | emulsifying surfactants | stable as it starts to |
| | | | and other oily | offset improvements |
| | | | components. Slowly add | in hand feel. |
| | | | to aqueous phase with | |
| | | | good agitation. Like | |
| | | | PDMS, increasing | |
| | | | emulsifier content | |
| | | | increases stability, but | |
| | | | also increases potential | |
| | | | for tackiness at dry | |
| | | | down. | |
| Dowsil EP 9801 | Provides a reduced- | 0.1-12.5% | Cold blend with | Provides good hand |
| Hydro Cosmetic | shine, mattifying | | remaining ingredients. | feel. |
| Powder | effect on the skin; | | Forms a stable | |
| (Dimethicone/Vinyl | sebum and oil | | suspension. | |
| Dimethicone | absorption; smooth, | | | |
| Crosspolymer (and) | powdery feel; and | | | |
| Silica (and) Butylene | spreadability and | | | |
| Glycol) | slipperiness. | | | |
| Fatty Esters and | Designed as | 0.5-1.0% | Blend esters with | Provides good hand |
| blends | replacements for | | emulsifiers and other | feel. |
| | PDMS. Ideally | | oily materials. Slowly | Emulsions formed |
| | easier to emulsify | | add to aqueous phase | showed evidence of |
| | than PDMS. | | with good agitation. | creaming over a week |
| | | | Increasing the amount of | at room temperature, |
| | | | emulsifier will tend to | but have not separated. |
| | | | increase the stability of | |
| | | | the emulsion, but at the | |
| | | | risk of the system feeling | |
| | | | “tacky” during dry-down. | |
| Esterquats (e.g., | Fatty quaternary | 1-8% | Add liquid versions | Provides good hand |
| Rewoquat WE 45) | compounds with | | directly to lotion. Forms | feel comparable to |
| | weak ester linkages | | a hazy emulsion stable at | lauroyl lysine. |
| | commonly used in | | room temperature for at | Liquid versions easier |
| | fabric softening, | | least a week. Solid | to blend than solids. |
| | hair care, and skin | | versions need to be | |
| | care. Cationic head | | melted before adding to | |
| | is attracted to | | heated base and allowed | |
| | negatively charged | | to cool. | |
| | surfaces, and fatty | | | |
| | tails give glide | | | |

For esterquats as an additive, in a discernment and preference screening, nine (9) out of nine (9) subjects could tell a difference between the 1% ester quat containing wipe and the base formula, and seven (7) out of nine (9) subjects preferred the ester quat version. The other two (2) subjects had no preference.

### Example 14: Panel Hand-Feel Data—Lauroyl Lysine Lotion Formulations (Personal Care Components—Skin Protectants)

This Example evaluates panel hand-feel data comparison for control lotion formulations and formulations including a lauroyl lysine additive.

A comparison between a control product and a product containing 1% lauroyl lysine was conducted with eight (8) subjects. Of the subjects, four (4) preferred the lauroyl lysine formulation, two (2) had no preference, and two (2) preferred the original because they thought that it dried faster. One of the subjects that preferred the base formulation advised that after their hands had dried completely, they still had some squeak or chatter from the base formulation but that the lauroyl lysine formulation felt smoother or moisturizing on their skin.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the systems and methods of the disclosed subject matter without departing from the spirit or scope of the disclosed subject matter. Thus, it is intended that the disclosed subject matter include modifications and variations that are within the scope of the appended claims and their equivalents.

Various patents and patent applications are cited herein, the contents of which are hereby incorporated by reference herein in their entireties.

What is claimed is:

1. A dispersible wipe comprising a fibrous material and a lotion formulation, wherein the lotion formulation has a dielectric constant of less than about 80.

2. The dispersible wipe of claim 1, wherein the lotion formulation comprises at least one solvent present in an amount of at least about 10 wt.-% and at least one salt present in an amount of at least about 0.5 wt.-%, based on the total weight of the lotion formulation.

3. The dispersible wipe of claim 2, wherein the at least one solvent comprises butylene glycol, hexylene glycol, or combinations thereof, and the at least one salt comprises calcium chloride.

4. The dispersible wipe of claim 2, wherein the lotion formulation comprises at least one solvent present in an amount of at least about 15 wt.-% with at least about 5 wt.-% hexylene glycol, based on the total weight of the lotion formulation.

5. The dispersible wipe of claim 2, wherein the lotion formulation comprises butylene glycol present in an amount of at least about 30 wt.-%, and the at least one salt comprises calcium chloride.

6. The dispersible wipe of claim 1, wherein the lotion formulation comprises at least one solvent miscible with water having a dielectric constant or a Hansen Solubility Parameter with a lower polarity than water.

7. The dispersible wipe of claim 1, wherein the lotion formulation comprises a solvent blend comprising at least one solvent miscible with water and at least one solvent immiscible with water, and wherein the solvent blend is miscible with water.

8. The dispersible wipe of claim 1, wherein the lotion formulation comprises a solvent blend comprising water and at least one solvent miscible with water.

9. The dispersible wipe of claim 1, wherein the lotion formulation comprises at least one solvent comprising phenols, monohydric alcohols, diols, polyhydric alcohols, unsaturated aliphatic alcohols, alicyclic alcohols, glycols, glycol ethers, glycerin, glycol ethers, 3 propanediol, acetone, acetonitrile, or combinations thereof.

10. The dispersible wipe of claim 6, wherein the at least one solvent comprises mineral oil, shea butter, cocoa butter, paraffin, beeswax, squalene, coconut oil, olive oil, cetyl alcohol, isopropyl myristate, triethylhexanoin, waxes, synthetic oils, plant oils, or combinations thereof.

11. The dispersible wipe of claim 1, wherein the lotion formulation comprises at least one solvent present in an amount of from about 5 wt. % to about 80 wt. %, based on the total weight of the lotion formulation.

12. The dispersible wipe of claim 1, wherein the lotion formulation comprises at least one coupling agent.

13. The dispersible wipe of claim 12, wherein the at least one coupling agent is a nonionic surfactant comprising polysorbate, alkylpolyglucosides, ethoxylated surfactants, sorbitan derivatives, betaines, amine oxides, or combinations thereof.

14. The dispersible wipe of claim 12, wherein the at least one coupling agent is present in an amount of from about 0.1 wt. % to about 10 wt. %, based on the total weight of the lotion formulation.

15. The dispersible wipe of claim 1, wherein the lotion formulation comprises at least one fragrance.

16. The dispersible wipe of claim 15, wherein the at least one fragrance is present in amount of from about 0 wt. % to about 0.5 wt. %, based on the total weight of the lotion formulation.

17. The dispersible wipe of claim 1, wherein the lotion formulation comprises at least one preservative.

18. The dispersible wipe of claim 17, wherein the at least one preservative is present in an amount of from about 0 wt. % to about 1.0 wt. %, based on the total weight of the lotion formulation.

19. The dispersible wipe of claim 1, wherein the lotion formulation comprises at least one skin protectant.

20. The dispersible wipe of claim 19, wherein the at least one skin protectant is present in an amount of from about 0 wt. % to about 10 wt. %, based on the total weight of the lotion formulation.

21. The dispersible wipe of claim 1, wherein the lotion formulation comprises at least one cationic disinfectant.

22. The dispersible wipe of claim 21, wherein the at least one cationic disinfectant is present in an amount of from about 0 wt. % to about 1.0 wt. %, based on the total weight of the lotion formulation.

23. The dispersible wipe of claim 21, wherein the at least one cationic disinfectant comprises alcohols, glycols, or a combination thereof present in an amount greater than about 50% by volume.

24. The dispersible wipe of claim 1, wherein the lotion formulation comprises at least one detergent.

25. The dispersible wipe of claim 24, wherein the at least one detergent is present in an amount of about 0 wt. % to about 20 wt. %, based on the total weight of the lotion formulation.

26. The dispersible wipe of claim 1, wherein the lotion formulation has a dielectric constant of between about 10 and about 60.

27. The dispersible wipe of claim 1, wherein the lotion formulation has a dielectric constant of between about 30 and about 80.

28. A dispersible wipe comprising a fibrous material, a water-soluble binder, and a lotion formulation, wherein the lotion formulation has a dielectric constant of less than about 80.

29. The dispersible wipe of claim 28, wherein the lotion formulation comprises at least one solvent present in an amount of at least about 15 wt.-% and at least one salt present in an amount of at least about 0.5 wt.-%, based on the total weight of the lotion formulation.

30. The dispersible wipe of claim 29, wherein the at least one solvent comprises butylene glycol and the at least one salt comprises calcium chloride.

31. The dispersible wipe of claim 29, wherein the lotion formulation comprises at least one solvent present in an amount of at least about 20 wt.-%, based on the total weight of the lotion formulation.

32. The dispersible wipe of claim 29, wherein the lotion formulation comprises butylene glycol present in an amount of at least about 20 wt.-% and the at least one salt comprises calcium chloride.

33. The dispersible wipe of claim 28, wherein the lotion formulation comprises at least one solvent miscible with water having a dielectric constant or a Hansen Solubility Parameter with a lower polarity than water.

34. The dispersible wipe of claim 28, wherein the lotion formulation comprises a solvent blend comprising at least one solvent miscible with water and at least one solvent immiscible with water, and wherein the solvent blend is miscible with water.

35. The dispersible wipe of claim 28, wherein the lotion formulation comprises a solvent blend comprising water and at least one solvent miscible with water.

36. The dispersible wipe of claim 28, wherein the lotion formulation comprises at least one solvent comprising phenols, monohydric alcohols, diols, polyhydric alcohols, unsaturated aliphatic alcohols, alicyclic alcohols, glycols, glycol ethers, glycerin, glycol ethers, 3 propanediol, acetone, acetonitrile, or combinations thereof.

37. The dispersible wipe of claim 33, wherein the at least one solvent comprises mineral oil, shea butter, cocoa butter, paraffin, beeswax, squalene, coconut oil, olive oil, cetyl alcohol, isopropyl myristate, triethylhexanoin, waxes, synthetic oils, plant oils, or combinations thereof.

38. The dispersible wipe of claim 28, wherein the lotion formulation comprises at least one solvent present in an amount of from about 5 wt. % to about 80 wt. %, based on the total weight of the lotion formulation.

39. The dispersible wipe of claim 28, wherein the lotion formulation comprises at least one coupling agent.

40. The dispersible wipe of claim 39, wherein the at least one coupling agent is present in an amount of from about 0.1 wt. % to about 5 wt. %, based on the total weight of the lotion formulation.

41. The dispersible wipe of claim 28, wherein the lotion formulation comprises at least one fragrance.

42. The dispersible wipe of claim 41, wherein the at least one fragrance is present in amount of from about 0 wt. % to about 0.5 wt. %, based on the total weight of the lotion formulation.

43. The dispersible wipe of claim 28, wherein the lotion formulation comprises at least one preservative.

44. The dispersible wipe of claim 43, wherein the at least one preservative is present in an amount of from about 0 wt. % to about 1.0 wt. %, based on the total weight of the lotion formulation.

45. The dispersible wipe of claim 28, wherein the lotion formulation comprises at least one skin protectant.

46. The dispersible wipe of claim 45, wherein the at least one skin protectant is present in an amount of from about 0 wt. % to about 10 wt. %, based on the total weight of the lotion formulation.

47. The dispersible wipe of claim 28, wherein the lotion formulation comprises at least one disinfectant.

48. The dispersible wipe of claim 47, wherein the at least one disinfectant is present in an amount of from about 0 wt. % to about 1.0 wt. %, based on the total weight of the lotion formulation.

49. The dispersible wipe of claim 47, wherein the at least one disinfectant comprises alcohols, glycols, or a combination thereof present in an amount greater than about 50% by volume.

50. The dispersible wipe of claim 28, wherein the lotion formulation comprises at least one detergent.

51. The dispersible wipe of claim 50, wherein the at least one detergent is present in an amount of about 0 wt. % to about 20 wt. %, based on the total weight of the lotion formulation.

52. The dispersible wipe of claim 28, wherein the lotion formulation comprises at least one salt.

53. The dispersible wipe of claim 52, wherein the at least one salt comprises a cation comprising sodium, potassium, calcium, magnesium, iron, aluminum, potassium, silver, tin, zinc, ammonium, or combinations thereof and anions selected from the group consisting of chloride, phosphate, sulfate, nitrite, or nitrate.

54. The dispersible wipe of claim 52, wherein the at least one salt is calcium chloride.

55. The dispersible wipe of claim 28, wherein the lotion formulation has a dielectric constant of between about 10 and about 60.

56. The dispersible wipe of claim 28, wherein the lotion formulation has a dielectric constant of between about 30 and about 80.

57. A lotion formulation suitable for use with fibrous substrates comprising at least one solvent, wherein the formulation has a dielectric constant of less than about 80.

58. A kit comprising a nonwoven substrate and a lotion formulation, wherein the lotion formulation has a dielectric constant of less than about 80.
